(19) United States
(12) Patent Application Publication
(10) Pub. No.: US 2013/0309306 A1
(43) Pub. Date: Nov. 21, 2013

(54) INTRAPULMONARY BENZODIAZEPINE FOR THE TREATMENT AND PREVENTION OF SEIZURES

(71) Applicants: Michael A. Rogawski, Sacramento, CA (US); Ashish Dhir, Sacramento, CA (US)

(72) Inventors: Michael A. Rogawski, Sacramento, CA (US); Ashish Dhir, Sacramento, CA (US)

(73) Assignee: THE REGENTS OF THE UNIVERSITY OF CALIFORNIA, Oakland, CA (US)

(21) Appl. No.: 13/885,660

(22) PCT Filed: Dec. 1, 2011
(86) PCT No.: PCT/US11/62888

§ 371 Date: Jul. 25, 2013

Related U.S. Application Data
(60) Provisional application No. 61/418,510, filed on Dec. 1, 2010.

(51) Int. Cl. A61K 9/00; A61K 31/5517

(52) U.S. Cl. CPC A61K 9/007; A61K 31/5517
USPC 424/489; 514/220

(57) ABSTRACT

This invention provides methods for the amelioration, termination and/or abortion of a seizure by the intrapulmonary administration of a benzodiazepine, for example, midazolam.

### CROSS REFERENCE TO RELATED APPLICATIONS

The present application claims the benefit of U.S. Provisional Application No. 61/418,510, filed on Dec. 1, 2010, which is hereby incorporated herein in its entirety for all purposes.

### FIELD OF THE INVENTION

The present invention relates to the intrapulmonary administration of midazolam for the treatment and prevention of seizures, for example, clonic and tonic seizures.

### BACKGROUND OF THE INVENTION

Inhaled therapeutic agents have been in clinical use for more than a century mainly for the treatment of pulmonary diseases such as asthma. The intrapulmonary route could be also used for delivering drugs systemically. The lungs offer a large absorptive area of approximately 100 m² presenting a minimal barrier for drug absorption. The intrapulmonary route has advantages over other conventional routes of systemic drug delivery such as avoidance of first pass metabolism, rapid systemic delivery as compared to intranasal or rectal route, and improved bioavailability as compared to the oral route. The blood exiting the lungs passes through the left heart and is directly carried to brain via the carotid circulation. Therefore, drug substances administered into the lung may affect the brain more rapidly than with other routes of administration. Other than volatile anesthetics, no other drug is marketed as an inhaler system to target central nervous system. Recently, there has been some research delivering anti-migraine drugs to the brain via an intrapulmonary delivery system (Aurora et al., 2009 Headache 49: 826-837).

Midazolam is a rapid onset, short acting water soluble benzodiazepine that is administered parenterally as a sedative, anxiolytic, hypnotic and amnestic agent. The drug is also administered intramuscularly, intravenously or intranasally to terminate acute seizures and status epilepticus (Galvin and Jelinek, 1987 Arch Emerg Med 4: 169-172). Midazolam exhibits anticonvulsant activity in diverse chemoconvulsant seizure models, including the pentylenetetrazol model in various species (Pieri, 1983 Br J Clin Pharmacol 16 Suppl 1: 17S-27S; Raines et al., 1990 Epilepsia 31: 313-317; Orebaugh and Bradford, 1994 Am J Emerg Med 12: 284-287; Jaimovich et al., 1990 Crit. Care Med 18: 313-316). As is the case for other benzodiazepines, the pharmacological actions of midazolam are mediated predominantly through an interaction with a high affinity binding site in brain representing an allosteric modulatory site on GABA_{A} receptors (Kucken et al., 2003 Mol Pharmacol 63: 289-296). Binding of midazolam and other classic benzodiazepines such as clonazepam to a recognition site at the interface between extracellular domains of the GABA_{A} receptor γ subunit and one of the α subunits allosterically modulates gating of the GABA_{A} receptor chloride channel complex leading to enhanced channel current (Rovira and Ben-Ari, 1999 J Neurophysiol 70: 1076-1085; Rüsch and Forman, 2005 Anesthesiology 102: 783-792; Eom et al., 2011 Korean J Anesthesiol 60: 109-118). This allosteric modulation of GABA_{A} receptors is believed to account for the principal pharmacological actions of benzodiazepines including their anticonvulsant activity (Rogawski, 2002 Principles of antiepileptic drug action. In, Levy R H, Mattson R H, Meldrum B S, Perucca E (eds). Antiepileptic Drugs, Fifth Edition, Lippincott Williams & Wilkins: Philadelphia, pp 3-22).

Some benzodiazepines also bind to a pharmacologically distinct and unrelated binding site in brain and peripheral tissues that was formerly described as the peripheral-type benzodiazepine receptor (Gavish et al., 1999 Pharmacol Rev 51: 629-650) but is now referred to as translocator protein (18 kDa) (TSPO) (Papadopoulos et al., 2006 Trends Pharmacol Sci 27: 402-409). TSPO is expressed predominantly in mitochondria, where it is localized to the outer mitochondrial membrane. TSPO binds cholesterol with high affinity and transports it from the outer mitochondrial membrane to the inner mitochondrial membrane, where it is converted to pregnenolone by cytochrome P450 side-chain cleavage enzyme (P450scc). This sequence represents the initial and rate-limiting step in the biosynthesis of all steroids. TSPO agonist ligands, including benzodiazepines with TSPO binding activity (Mukhin et al., 1989 Proc Natl Acad Sci USA 86: 9813-9816), stimulate steroidogenesis by facilitating cholesterol delivery to P450scc in the inner mitochondrial membrane (Lacapere and Papadopoulos, 2003 Steroids 68: 569-585).

As is the case for hormonal steroids, a variety of evidence supports the concept that TSPO agonist ligands can also enhance the synthesis of endogenous neurosteroids, including allopregnanolone, that lack hormonal activity but serve as positive allosteric modulators of GABA_{A} receptors (Romeo et al., 1993 J Pharmacol Exp Ther 267: 462-471; Serra et al., 1999 Br J Pharmacol 127: 177-187; Bitran et al., 2000 Psychopharmacology (Berl) 151: 64-71; Verleye et al., 2005 Pharmacol Biochem Behav 82: 712-720). In accordance with their effects on GABA_{A} receptors, such neurosteroids exhibit anticonvulsant activity in various seizure models, including PTZ-induced seizures in mice (Kokate et al., 1994 J Pharmacol Exp Ther. 270: 1223-1229). The effect of TSPO agonist ligands on neurosteroidogenesis is believed to occur through enhanced mitochondrial synthesis of pregnenolone, which is converted to progesterone by microsomal 3β-hydroxysteroid dehyrogenase and then to neurosteroids by sequential A-ring reduction by 5α-reductase and 3α-hydroxysteroidoxidoreductase. It has been demonstrated that TSPO ligand-induced enhanced endogenous neurosteroid synthesis can produce pharmacological effects typical of neurosteroids, including anxiolytic actions (Kita and Furukawa, 2005 Pharmacol Biochem Behav 89: 171-178; Rupprecht et al., 2009 Science 325: 490-493). In some experimental situations, the enhanced neurosteroid synthesis (Serra et al., 1999 Br J Pharmacol 127: 177-187; Frye et al., 2009 Reproduction 137: 119-128) and behavioral effects (Bitran et al., 2000 Psychopharmacology (Berl) 151: 64-71; Kita et al., 2004 Br J Pharmacol 142: 1059-1072; Rupprecht et al., 2009 Science 325: 490-493) of TSPO ligands can be reduced by the isoquinoline carboxamide TSPO antagonist PK 11195. Although there is some evidence that midazolam interacts with TSPO (Schoemaker et al., 1981 Eur J Pharmacol 71: 173-175; Bender and Hertz, 1987 J Neurosci Res 18: 366-372; Mak and Barnes, 1990 J Pharmacol Exp Ther 252: 880-885; Matsumoto et al., 1994 Antimicrob Agents Chemother 38: 812-816), until recently it was not known if midazolam acts as a functional TSPO ligand to affect steroidogenesis. However, So et al. (2010 Toxicol Lett 192: 169-178) have now reported that midazolam enhances Leydig cell production of progesterone and testosterone. In addition, Tokuda et al. (2010 J Neurosci 30: 16788-16795) observed that midazolam enhances the synthesis of neurosteroids in hippocampal neurons in brain slices. These latter authors presented evidence that neurosteroidogenesis in addition to an interaction with the benzodiazepine binding site on GABA_{A} receptors is required for the actions of midazolam on synaptic inhibition.

### SUMMARY OF THE INVENTION

In one aspect, the present invention provides methods of preventing or terminating a seizure in a subject in need thereof, comprising intrapulmonary administration to the subject of an effective amount of a benzodiazepine receptor agonist. The agonist can be a benzodiazepine or a non-benzodiazepine.

In a related aspect, the invention provides methods of accelerating the termination or abortion of an impending seizure in a subject in need thereof, comprising intrapulmonary administration to the subject of an effective amount of a benzodiazepine. Termination or abortion of the seizure is accelerated and improved, e.g., in comparison to termination or abortion of the seizure by administration of the benzodiazepine by intravenous or intranasal administration.

In some embodiments, the benzodiazepine is a positive allosteric modulator (e.g., an agonist) of GABA_{A} receptors and stimulates endogenous neurosteroid synthesis.

In some embodiments, the benzodiazepine is a positive allosteric modulator of GABA_{A} receptors and an agonist of peripheral benzodiazepine receptors (PBRs). In some embodiments, the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the benzodiazepine is midazolam.

In some embodiments, the subject is experiencing an aura, e.g., that indicates an impending or imminent seizure. In some embodiments, the subject has been warned of an impending seizure, e.g., by a seizure warning or detection system. The seizure detection system may be attached to the skin of the subject or implanted in the subject.

In some embodiments, the subject is experiencing a seizure.

In some embodiments the subject has status epilepticus. In some embodiments the subject has myoclonic epilepsy. In some embodiments, the subject suffers from seizure clusters. In some embodiments, the seizure is a tonic seizure. In some embodiments, the seizure is a clonic seizure.

In some embodiments, the benzodiazepine is administered via an inhaler. In some embodiments, the benzodiazepine is nebulized or aerosolized. In some embodiments, the benzodiazepine is nebulized or aerosolized without heating. In some embodiments, the nebulized or aerosolized benzodiazepine particles have a mass median aerodynamic diameter (“MMAD”) of about 3 μm or smaller. In some embodiments, the nebulized or aerosolized benzodiazepine particles have a mass median aerodynamic diameter (“MMAD”) of about 2-3 μm. In some embodiments, the benzodiazepine is delivered to the distal alveoli.

In a related aspect, the present invention provides methods of preventing or terminating a seizure in a subject in need thereof, comprising intrapulmonary administration to the subject of an effective amount of midazolam. In another aspect, the invention provides methods of accelerating the termination or abortion of an impending seizure in a subject in need thereof, comprising intrapulmonary administration to the subject of an effective amount of midazolam. In some embodiments, the midazolam is administered via an inhaler. In some embodiments, midazolam is dissolved in an aqueous solution that is nebulized or aerosolized. In some embodiments, the midazolam is nebulized or aerosolized without heating. In some embodiments, the nebulized or aerosolized midazolam particles have a mass median aerodynamic diameter (“MMAD”) of about 5 μm, 4 μm, 3 μm or smaller. In some embodiments, the nebulized or aerosolized midazolam particles have a mass median aerodynamic diameter (“MMAD”) of about 2-3 μm. In some embodiments, the midazolam is delivered to the distal alveoli. In some embodiments, the midazolam is administered at a dose in the range of about 0.3 μg/kg to about 3.0 μg/kg. In some embodiments, the midazolam is administered at a dose that is less than about 3.5 μg/kg. In some embodiments, the midazolam is administered at a dose in the range of about 3.0 μg/kg to about 25 μg/kg.

In some embodiments, the benzodiazepine is administered at a dose in the range of about 0.3 μg/kg to about 3.0 μg/kg. In some embodiments, the benzodiazepine is administered at a dose that is less than about 3.5 μg/kg. In some embodiments, the benzodiazepine is administered at a dose in the range of about 3.0 μg/kg to about 25 μg/kg.

In some embodiments, the benzodiazepine is self-administered by the subject. In some embodiments, the benzodiazepine is administered by a caregiver who is not the subject.

In some embodiments, the benzodiazepine is co-administered with a neurosteroid. The neurosteroid can also be administered via the intrapulmonary route.

In some embodiments, the subject is a human.

In a further aspect, the present invention provides methods of preventing or terminating a seizure in a subject in need thereof, comprising intrapulmonary administration to the subject of an effective amount of a neurosteroid.

In a related aspect, the invention provides methods of accelerating the termination or abortion of an impending seizure in a subject in need thereof, comprising intrapulmonary administration to the subject of an effective amount of a neurosteroid. Termination or abortion of the seizure is accelerated and improved, e.g., in comparison to termination or abortion of the seizure by administration of the neurosteroid by intravenous or intranasal administration.

### DEFINITIONS

The term “co-administration” refers to the presence of both active agents in the blood at the same time. Active agents that are co-administered can be delivered concurrently (i.e., at the same time) or sequentially.

The terms “patient,” “subject” or “individual” interchangeably refers to a mammal, for example, a human or a non-human mammal, including primates (e.g., macaque, pan troglodyte, pongo), a domesticated mammal (e.g., felines, canines), an agricultural mammal (e.g., bovine, ovine, porcine, equine) and a laboratory mammal or rodent (e.g., rattus, murine, lagomorpha, hamster).

The terms “reduce,” “inhibit,” “relieve,” “alleviate” refer to the detectable decrease in the frequency, severity and/or duration of seizures. A reduction in the frequency, severity and/or duration of seizures can be measured by self-assessment (e.g., by reporting of the patient) or by a trained clinical observer. Determination of a reduction of the frequency, severity and/or duration of seizures can be made by comparing patient status before and after treatment.

The term “effective amount” or “pharmaceutically effective amount” refer to the amount and/or dosage, and/or dosage regime of one or more compounds necessary to bring about the desired result e.g., an amount sufficient prevent, abort or terminate a seizure.

The phrase “cause to be administered” refers to the actions taken by a medical professional (e.g., a physician), or a person controlling medical care of a subject, that control and/or permit the administration of the agent(s)/compound(s) at issue to the subject. Causing to be administered can involve diagnosis and/or determination of an appropriate therapeutic or prophylactic regimen, and/or prescribing particular agent(s)/compounds for a subject. Such prescribing can include, for example, drafting a prescription form, annotating a medical record, and the like.

### BRIEF DESCRIPTION OF THE DRAWINGS

FIG. 1 illustrates the effect of intraperitoneal (A; 100-5000 μg/kg) and intratracheal (B; 12.5-100 μg/kg) midazolam on myoclonic jerk, generalized clonus and tonic extension threshold in response to intravenous PTZ infusion in mice. Midazolam was administered 15 and 10 minutes in case of intraperitoneal and intratracheal experiments, respectively, prior to the beginning of the PTZ infusion. Bars indicate mean±S.E.M. of values from 6-8 mice normalized with respect to the thresholds in the vehicle-treated groups (V)). *P<0.001 as compared to vehicle control group (ANOVA followed by Tukey's test).

FIG. 2 illustrates the time course for the action of 100 μg/kg intratracheal midazolam on myoclonic jerk, generalized clonus and tonic extension threshold in response to intravenous PTZ infusion in mice. Midazolam was administered 5, 10, 20, 40, 60 and 120 min prior to the beginning of the PTZ infusion. Closed (▪) and open (□) symbols indicate mean±S.E.M. of values from 6-8 mice pretreated with midazolam or vehicle, respectively). *P<0.05 as compared to vehicle control group (ANOVA followed by Tukey's test).

FIG. 3 illustrates the effect of intraperitoneal (A; 100-1000 μg/kg) or intratracheal (B; 25 and 50 μg/kg) midazolam on myoclonic jerk, generalized clonus and tonic extension induced in response to intravenous picrotoxin infusion in mice. Midazolam was administered 10 min prior to the beginning of the picrotoxin infusion for both intraperitoneal and intratracheal experiments. Bars indicate mean±S.E.M. of values from 6-8 mice normalized with respect to the threshold in the vehicle-treated groups (V). *P<0.05 as compared to vehicle control group (ANOVA followed by Tukey's test).

FIG. 4 illustrates the effect of intraperitoneal (A; 1000 and 2000 μg/kg) and intratracheal (B; 100 μg/kg) administration of midazolam on myoclonic jerk, generalized clonus and tonic extension induced in response to intravenous kainic acid infusion in mice. Midazolam was administered 10 min prior to the beginning of the kainic acid infusion. Bars indicate mean±S.E.M. of values from 6-8 mice normalized with respect to the threshold in the vehicle-treated group. *P<0.05 as compared to vehicle control group (ANOVA followed by Tukey's test).

FIG. 5 illustrates an intravenous (i.v.) midazolam and PTZ i.v. seizure threshold model-dose response curve. The results demonstrate the unexpectedly superior potency of intrapulmonary midazolam in inhibiting PTZ-induced seizures in mice in comparison to midazolam delivered via the intravenous or intraperitoneal route. Midazolam was administered 10 minutes before challenging the animals with intravenous PTZ. Whereas midazolam was active as anticonvulsant at doses of 25 μg/kg and 50 μg/kg when administered intratracheally, higher doses of 100-200 μg/kg were required when administered intravenously; a dose of 500 μg/kg was required when administered intraperitoneally.

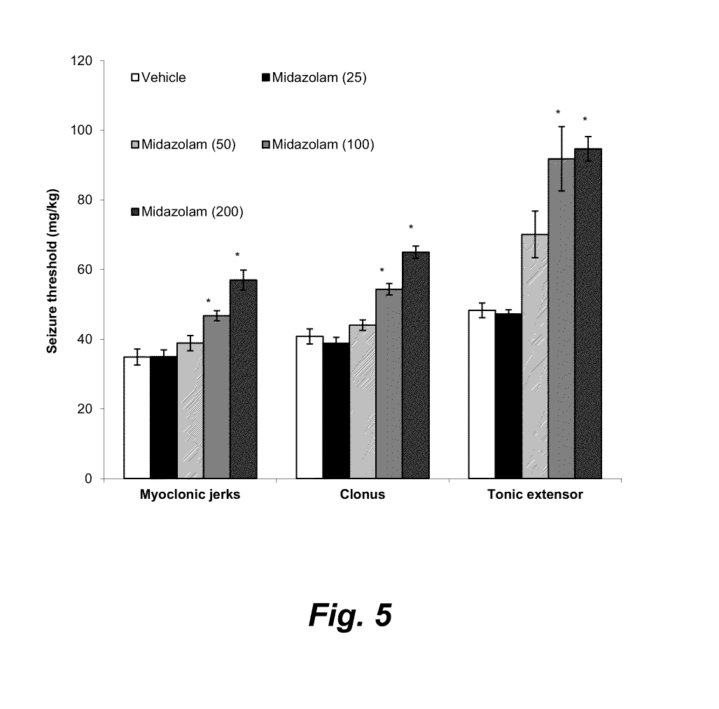

FIG. 6 illustrates drug treatment protocols. In some experiments, clonazepam (25 or 100 μg/kg, i.p.) was used instead of midazolam.

FIG. 7 illustrates dose-response relationship for midazolam protection against tonic extension in response to intravenous PTZ infusion in mice. Midazolam was administered intraperitoneally 15 min prior to the beginning of the PTZ infusion. Data points indicate mean±S.E.M. of threshold values from 6-8 mice normalized with respect to the mean threshold value in the vehicle-treated control group, which was 51.5±4.0 mg/kg. Dashed lines indicate the limits of the S.E.M. for the vehicle group. The mean threshold values for all groups other than the 100 μg/kg group are significantly different from the value in the vehicle group (*, p<0.001; one-way ANOVA followed by Tukey's test).

FIG. 8 illustrates that finasteride pretreatment reduces the seizure threshold elevation induced by midazolam. Finasteride (100 mg/kg, i.p.) or vehicle was administered 5 min before the treatment with midazolam (500 μg/kg, i.p.) or vehicle; 15 min after the second pretreatment, all animals were infused with PTZ. Bars indicate mean±S.E.M. of fractional threshold change values for tonic extension from 6-14 mice normalized with respect to the mean threshold value in the vehicle only control group, which was 59.2±1.8 mg/kg. In the absence of finasteride, midazolam caused a 2.3-fold increase in threshold (p<0.001). Finasteride did not reduce the threshold significantly in the absence of midazolam (NS) but did reduce the threshold with midazolam pretreatment (*, p<0.001). Statistical comparisons were made with one-way ANOVA followed by Tukey's test.

FIG. 9 illustrates metyrapone elevates seizure threshold in the absence and presence of midazolam. Metyrapone (100 mg/kg, i.p.) or vehicle was administered 15 min before treatment with midazolam or vehicle; 15 min after the second pretreatment, all animals were infused with PTZ. Bars indicate mean±S.E.M. of fractional change values in tonic extension threshold from 6-9 mice normalized with respect to the mean threshold value in the vehicle only control group, which was 51.0±4.4 mg/kg. *, p<0.001 as compared to vehicle only control group; •, p<0.001 as compared to midazolam only group. Statistical comparisons were made with one-way ANOVA followed by Tukey's test.

FIG. 10 illustrates PK 11195 pretreatment reduces the seizure threshold elevation induced by midazolam. PK11195 (15 mg/kg, i.p.) was administered 30 min before the treatment with midazolam (500 μg/kg, i.p.) or vehicle; 15 min after the second pretreatment, all animals were infused with PTZ. Bars indicate mean±S.E.M. of fractional threshold change values for tonic extension from 6-12 mice normalized with respect to the mean threshold value in the vehicle only control group (same as FIG. 9). *, p<0.001 as compared to vehicle only control group; •, p<0.001 as compared to midazolam only group. Statistical comparisons were made with one-way ANOVA followed by Tukey's test.

FIG. 11 illustrates finasteride (top panel) but not PK 11195 (bottom panel) pretreatment reduces the seizure threshold elevation induced by clonazepam. Finasteride (100 mg/kg, i.p.) or vehicle was administered 5 min before the treatment with clonazepam (100 μg/kg, i.p.) or vehicle; 15 min after the second pretreatment, all animals were infused with PTZ. In the absence of finasteride, clonazepam caused a 2.8-fold increase in threshold (p<0.001). Finasteride did not reduce the threshold significantly in the absence of midazolam (NS) but did reduce the threshold with clonazepam pretreatment (p<0.001). PK11195 (15 mg/kg, i.p.) was administered 30 min before the treatment with clonazepam or vehicle; 15 min after the second pretreatment, all animals were infused with PTZ. Bars indicate mean±S.E.M. of fractional threshold change values for tonic extension from 6-13 mice normalized with respect to the mean threshold value in the vehicle only control group, which was 46.9±3.6 mg/kg in the experiment with finasteride and 51.9±2.7 mg/kg in the experiment with PK 11195. *, p<0.001 as compared to vehicle only control group; •, p<0.001 as compared to clonazepam only group. Statistical comparisons were made with one-way ANOVA followed by Tukey's test.

FIG. 12 illustrates that flumazenil pretreatment abolishes the seizure threshold elevation induced by intratracheal administration of midazolam. Flumazenil (5 mg/kg, i.p.) or vehicle was administered 5 min before the treatment with midazolam (100 or 200 μg/kg, intratracheal) or vehicle; 10 min after the second pretreatment, all animals were infused with PTZ. Bars indicate mean±S.E.M. of seizure threshold in mg/kg from 6 mice. In the absence of flumazenil, midazolam (100 or 200 μg/kg, intratracheal) caused a significant increase in the seizure threshold. Flumazenil (5 mg/kg, i.p.) did not reduce the threshold significantly in the absence of midazolam but did reduce the threshold with midazolam pretreatment (100 or 200 μg/kg, intratracheal). *P<0.05 as compared to vehicle treated group treated (vehicle flumazenil+vehicle midazolam); $P<0.05 as compared to flumazenil per se (flumazenil+vehicle midazolam) group; #P<0.05 as compared to midazolam 100 μg/kg group (midazolam+vehicle flumazenil); @P<0.05 as compared to midazolam 200 μg/kg group (midazolam+vehicle flumazenil). Statistical comparisons were made with one-way ANOVA followed by Tukey's test.

FIG. 13 illustrates that finasteride pretreatment reduces the seizure threshold elevation induced by intratracheal administration of midazolam. Finasteride (50 and 100 mg/kg, i.p.) or vehicle was administered 5 min before the treatment with midazolam (100 μg/kg, intratracheal) or vehicle; 10 min after the second pretreatment, all animals were infused with PTZ. Bars indicate mean±S.E.M. of seizure threshold in mg/kg from 6 mice. In the absence of finasteride, midazolam (100 μg/kg, intratracheal) caused a significant increase in the seizure threshold. Finasteride (100 mg/kg, i.p.) did not reduce the threshold significantly in the absence of midazolam but did reduce the threshold with midazolam pretreatment. *P<0.05 as compared to vehicle treated (vehicle finasteride+vehicle midazolam) group; $P<0.05 as compared to finasteride per se (finasteride+vehicle midazolam) group; #P<0.05 as compared to midazolam (100 μg/kg., i.t.) group (midazolam+vehicle finasteride). Statistical comparisons were made with one-way ANOVA followed by Tukey's test.

### DETAILED DESCRIPTION

1. Introduction

The present invention is based, in part, on the recognition that benzodiazepines, including midazolam, and non-benzodiazepine benzodiazepine receptor agonists can be anticonvulsant agents. It is further based on the unexpected discovery that certain benzodiazepines can have greater and more effective anticonvulsant activity than other benzodiazepines as a result of their ability to enhance endogenous biosynthesis of neurosteroids (neurosteroidogenesis), which themselves are also anticonvulsant agents.

Midazolam is a short-acting benzodiazepine that is widely used as an intravenous sedative and anticonvulsant. Besides interacting with the benzodiazepine site associated with GABA_{A} receptors, some benzodiazepines act as agonists of translocator protein (18 kDa) (TSPO) to enhance the synthesis of steroids, including neurosteroids with positive modulatory actions on GABA_{A} receptors. We determined that neurosteroidogenesis induced by midazolam contributes to its anticonvulsant action.

Mice were pretreated with neurosteroid synthesis inhibitors and potentiators followed by midazolam or clonazepam, a weak TSPO ligand. Anticonvulsant activity was assessed with the intravenous pentylenetetrazol (PTZ) threshold test, an animal model representative of myoclonic epilepsy. The PTZ seizure model demonstrated herein is predictive of utility and/or activity in counteracting myoclonic seizures or myoclonic epilepsy in humans.

Midazolam (500-5000 μg/kg, i.p.) caused a dose-dependent increase in seizure threshold. Pretreatment with the neurosteroid synthesis inhibitors finasteride, a 5α-reductase inhibitor, and 1-(2-chlorophenyl)-N-methyl-N-(1-methylpropyl)-3-isoquinolinecarboxamide (PK 11195), a functional TSPO antagonist, reduced the anticonvulsant action of midazolam. The anticonvulsant action of midazolam was enhanced by the neurosteroidogenic drug metyrapone, an 11β-hydroxylase inhibitor. In contrast, the anticonvulsant action of clonazepam (100 μg/kg) was reduced by finasteride but not by PK 11195, indicating a possible contribution of neurosteroids unrelated to TSPO.

Enhanced endogenous neurosteroid synthesis, likely mediated by an interaction with TSPO, contributes to the anticonvulsant action of midazolam. Enhanced neurosteroidogenesis may also be a factor in the actions of other benzodiazepines, even those that only weakly interact with TSPO.

The present invention provides a means for a practitioner skilled in the art to select from among available benzodiazepines and non-benzodiazepine benzodiazepine receptor agonists as preferred agents to ameliorate, terminate and/or abort a seizure. An example of such a preferred agent is midazolam, which has unexpectedly been found to protect against seizures by a dual mechanism involving (1) binding to the benzodiazepine recognition site on GABA_{A} receptors thus causing positive allosteric modulation of the GABA_{A} receptors, and (2) enhancement of neurosteroidogenesis leading to increased brain availability of neurosteroids which separately bind to a distinct (neurosteroid) recognition site on GABA_{A} receptors, thus directly activating and causing further positive modulation of the GABA_{A} receptors. The amount of activation and positive modulation of GABA_{A} receptors achieved by this dual mechanism is greater than the amount that is achieved by agents that only act by the first mechanism typical of the broad universe of benzodiazepines and non-benzodiazepine benzodiazepine receptor agonists. Agents such as midazolam have the dual mechanism of positively modulating GABA_{A} receptors and promoting neurosteroidogenesis. The increased anticonvulsant activity resulting from the action on benzodiazepine receptors and the distinct action to enhance neurosteroidogenesis provides superior activity in the prevention or treatment of seizures.

The present invention is further based, in part, on the unexpected discovery that intrapulmonary delivery of aqueous, non-heated formulations of appropriate benzodiazepines and non-benzodiazepine benzodiazepine receptor agonists more rapidly protects against and terminates seizures than occurs when the same agents are administered by other routes. These agents, including midazolam (a short acting benzodiazepine), find use for formulation for intrapulmonary administration, e.g., via inhaler.

Midazolam has been administered intramuscularly or intravenously for terminating acute seizure attacks or status epilepticus (Galvin and Jelinek, 1987). Midazolam has shown anticonvulsant activity in a variety of seizure models (Orebaugh and Bradford, 1994, Jaimovich et al., 1990, Czlonkowska et al., 2001, Raines et al., 1990, Liefaard et al., 2007). Midazolam specifically binds to the benzodiazepine recognition site of the GABA_{A} receptor and thus facilitates the inhibitory effect of GABA by increasing the frequency of opening of the intrinsic chloride ion channel (Wieland et al., 1992). Recently, nasal midazolam has gained an important attention as a novel, rapid and convenient route of application against seizure attacks (Bhattacharyya et al., 2006; Gilat et al., 2003). However, the present invention is based, in part, on the discovery that intrapulmonary administration of midazolam and other anticonvulsant benzodiazepines and non-benzodiazepine benzodiazepine receptor agonists provides a more rapid means of drug delivery to the brain as compared to intranasal delivery. This allows seizures to be terminated or aborted during the brief period of the aura. It also allows status epilepticus and seizure clusters to be terminated or aborted more rapidly, resulting in the reduced possibility of physical injury from continuing seizures and a greater prevention from the progressive brain damage that occurs with continuing status epilepticus.

An inhaler system of an anticonvulsant benzodiazepine, e.g., midazolam, finds use for administration to epileptic patients to ameliorate or terminate acute seizure attacks during the aura phase of temporal lobe epilepsy and to ameliorate or abort status epilepticus, a severe condition where seizures do not terminate spontaneously and continue for ten minutes or more. The inhaler system also finds use along with a seizure prediction device.

2. Conditions Amenable to Treatment

Intrapulmonary administration of a benzodiazepine, including midazolam, or a non-benzodiazepine benzodiazpeine receptor agonist, finds use in the rapid amelioration and/or termination of seizures. In various embodiments, the seizures may be due to an epileptic condition.

The term “epilepsy” refers to a chronic neurological disorder characterized by recurrent unprovoked seizures. These seizures are transient signs and/or symptoms of abnormal, excessive or synchronous neuronal activity in the brain. There are over 40 different types of epilepsy, including without limitation childhood absence epilepsy, juvenile absence epilepsy, benign Rolandic epilepsy, clonic seizures, complex partial seizures, frontal lobe epilepsy, febrile seizures, infantile spasms, juvenile myoclonic epilepsy, Lennox-Gastaut syndrome, Landau-Kleffner Syndrome, myoclonic seizures, mitochondrial disorders associated with seizures, Lafora Disease, progressive myoclonic epilepsies, reflex epilepsy, and Rasmussen's syndrome. There are also numerous types of seizures including simple partial seizures, complex partial seizures, generalized seizures, secondarily generalized seizures, temporal lobe seizures, tonic-clonic seizures, tonic seizures, psychomotor seizures, limbic seizures, status epilepticus, abdominal seizures, akinetic seizures, autonomic seizures, massive bilateral myoclonus, drop seizures, focal seizures, gelastic seizures, Jacksonian march, motor seizures, multifocal seizures, neonatal seizures, nocturnal seizures, photosensitive seizure, sensory seizures, sylvan seizures, withdrawal seizures and visual reflex seizures.

The most widespread classification of the epilepsies divides epilepsy syndromes by location or distribution of seizures (as revealed by the appearance of the seizures and by EEG) and by cause. Syndromes are divided into localization-related epilepsies, generalized epilepsies, or epilepsies of unknown localization. Localization-related epilepsies, sometimes termed partial or focal epilepsies, arise from an epileptic focus, a small portion of the brain that serves as the irritant driving the epileptic response. Generalized epilepsies, in contrast, arise from many independent foci (multifocal epilepsies) or from epileptic circuits that involve the whole brain. Epilepsies of unknown localization remain unclear whether they arise from a portion of the brain or from more widespread circuits.

Epilepsy syndromes are further divided by presumptive cause: idiopathic, symptomatic, and cryptogenic. Idiopathic epilepsies are generally thought to arise from genetic abnormalities that lead to alterations in brain excitability. Symptomatic epilepsies arise from the effects of an epileptic lesion, whether that lesion is focal, such as a tumor, or a defect in metabolism causing widespread injury to the brain. Cryptogenic epilepsies involve a presumptive lesion that is otherwise difficult or impossible to uncover during evaluation. Forms of epilepsy are well characterized and reviewed, e.g., in Epilepsy: A Comprehensive Textbook (3-volume set), Engel, et al., editors, 2nd Edition, 2007, Lippincott, Williams and Wilkins; and The Treatment of Epilepsy: Principles and Practice, Wyllie, et al., editors, 4th Edition, 2005, Lippincott, Williams and Wilkins; and Browne and Holmes, Handbook of Epilepsy, 4th Edition, 2008, Lippincott, Williams and Wilkins.

3. Subjects Amenable to Treatment

In various embodiments, the patient may be experiencing an electrographic or behavioral seizure or may be experiencing a seizure aura, which itself is a localized seizure that may spread and become a full blown behavioral seizure. For example, the subject may be experiencing aura that alerts of the impending onset of a seizure or seizure cluster.

Alternatively, the subject may be using a seizure prediction device that alerts of the impending onset of a seizure or seizure cluster. Implantable seizure prediction devices are known in the art and described, e.g., in D'Alessandro, et al., IEEE TRANSACTIONS ON BIOMEDICAL ENGINEERING, VOL. 50, NO. 5, MAY 2003, and U.S. Patent Publication Nos. 2010/0198098, 2010/0168603, 2009/0062682, and 2008/0243022.

The subject may have a personal or familial history of any of the epileptic conditions described herein. The subject may have been diagnosed as having any of the epileptic conditions described herein. In some embodiments, the subject has or is at risk of suffering a myoclonic seizure or myoclonic epilepsy, e.g., juvenile myoclonic epilepsy. The PTZ seizure model demonstrated herein is predictive of utility and/or activity in counteracting myoclonic seizures or myoclonic epilepsy in humans.

In various embodiments, the subject may be at risk of exposure to or may have been exposed to a nerve agent or a pesticide that can cause seizures. Illustrative nerve agents that can cause seizures include, e.g., organophosphorus nerve agents, e.g., tabun, sarin, soman, GF, VR and/or VX. Illustrative pesticides that can cause seizures include, e.g., organophosphate pesticides (e.g., Acephate (Orthene), Azinphos-methyl (Gusathion, Guthion), Bensulide (Betasan, Lescosan), Bomyl (Swat), Bromophos (Nexion), Bromophos-ethyl (Nexagan), Cadusafos (Apache, Ebufos, Rugby), Carbophenothion (Trithion), Chlorethoxyfos (Fortress), Chlorfenvinphos (Apachlor, Birlane), Chlormephos (Dotan), Chlorphoxim (Baythion-C), Chlorpyrifos (Brodan, Dursban, Lorsban), Chlorthiophos (Celathion), Coumaphos (Asuntol, Co-Ral), Crotoxyphos (Ciodrin, Cypona), Crufomate (Ruelene), Cyanofenphos (Surecide), Cyanophos (Cyanox), Cythioate (Cyflee, Proban), DEF (De-Green), E-Z-Off D), Demeton (Systox), Demeton-5-methyl (Duratox, Metasystoxl), Dialifor (Torak), Diazinon, Dichlorofenthion, (VC-13 Nemacide), Dichlorvos (DDVP, Vapona), Dicrotophos (Bidrin), Dimefos (Hanane, Pestox XIV), Dimethoate (Cygon, DeFend), Dioxathion (Delnav), Disulfoton (Disyston), Ditalimfos, Edifenphos, Endothion, EPBP (S-seven), EPN, Ethion (Ethanox), Ethoprop (Mocap), Ethyl parathion (E605, Parathion, thiophos), Etrimfos (Ekamet), Famphur (Bash, Bo-Ana, Famfos), Fenamiphos (Nemacur), Fenitrothion (Accothion, Agrothion, Sumithion), Fenophosphon (Agritox, trichloronate), Fensulfothion (Dasanit), Fenthion (Baytex, Entex, Tiguvon), Fonofos (Dyfonate, N-2790), Formothion (Anthio), Fosthietan (Nem-A-Tak), Heptenophos (Hostaquick), Hiometon (Ekatin), Hosalone (Zolone), IBP (Kitazin), Iodofenphos (Nuvanol-N), Isazofos (Brace, Miral, Triumph), Isofenphos (Amaze, Oftanol), Isoxathion (E-48, Karphos), Leptophos (Phosvel), Malathion (Cythion), Mephosfolan (Cytrolane), Merphos (Easy Off-D, Folex), Methamidophos (Monitor), Methidathion (Supracide, Ultracide), Methyl parathion (E601, Penncap-M), Methyl trithion, Mevinphos (Duraphos, Phosdrin), Mipafox (Isopestox, Pestox XV), Monocrotophos (Azodrin), Naled (Dibrome), Oxydemeton-methyl (Metasystox-R), Oxydeprofos (Metasystox-S), Phencapton (G 28029), Phenthoate (Dimephenthoate, Phenthoate), Phorate (Rampart, Thimet), Phosalone (Azofene, Zolone), Phosfolan (Cylan, Cyolane), Phosmet (Imidan, Prolate), Phosphamidon (Dimecron), Phostebupirim (Aztec), Phoxim (Baythion), Pirimiphos-ethyl (Primicid), Pirimiphos-methyl (Actellic), Profenofos (Curacron), Propetamphos (Safrotin), Propyl thiopyrophosphate (Aspon), Prothoate (Fac), Pyrazophos (Afugan, Curamil), Pyridaphenthion (Ofunack), Quinalphos (Bayrusil), Ronnel (Fenchlorphos, Korlan), Schradan (OMPA), Sulfotep (Bladafum, Dithione, Thiotepp), Sulprofos (Bolstar, Helothion), Temephos (Abate, Abathion), Terbufos (Contraven, Counter), Tetrachlorvinphos (Gardona, Rabon), Tetraethyl pyrophosphate (TEPP), Triazophos (Hostathion), and Trichlorfon (Dipterex, Dylox, Neguvon, Proxol).

4. Therapeutic Agents

The methods involve the intrapulmonary administration of a benzodiazepine or a non-benzodiazepine benzodiazepine receptor agonist. Such agents that find use have anticonvulsant activity. In various embodiments, the agent concurrently is an agonist of the benzodiazepine recognition site (receptor) on GABA_{A} receptors and stimulates the synthesis of endogenous neurosteroids. In various embodiments, the agent concurrently is an agonist of the benzodiazepine receptor on GABA_{A} receptors and an agonist of peripheral benzodiazepine receptors or translocator protein 18 kD (TSPO).

The terms “neuroactive steroid” or “neurosteroids” interchangeably refer to steroids that rapidly alter neuronal excitability through interaction with neurotransmitter-gated ion channels, specifically GABA_{A} receptors. Neuroactive steroids have a wide range of applications from sedation to treatment of epilepsy and traumatic brain injury. Neuroactive steroids act as direct agonists and allosteric positive modulators of GABA_{A} receptors. Several synthetic neuroactive steroids have been used as sedatives for the purpose of general anaesthesia for carrying out surgical procedures. Exemplary sedating neuroactive steroids include without limitation alphaxolone, alphadolone, hydroxydione and minaxolone. The neuroactive steroid ganaxolone finds use for the treatment of epilepsy. In various embodiments, the benzodiazepine or non-benzodiazepine benzodiazepine receptor agonist is co-administered with an endogenously occurring neurosteroid or other neuroactive steroid. Illustrative endogenous neuroactive steroids, e.g., allopregnanolone and tetrahydrodeoxycorticosterone find use. In some embodiments, the neurosteroid is selected from the group consisting of allopregnanolone, allotetrahydrodeoxycorticosterone, ganaxolone, alphaxolone, alphadolone, hydroxydione, minaxolone, and Althesin.

Other neurosteroids of use include without limitation allotetrahydrodeoxycorticosterone (3α,21-dihydroxy-5α-pregnan-20-one; THDOC), 3α,21-dihydroxy-5b-pregnan-20-one, pregnanolone (3α-hydroxy-5β-pregnan-20-one), Ganaxolone (INN, also known as CCD-1042; IUPAC name (3α,5α)-3-hydroxy-5-methylpregnan-20-one; 1-[(3R,5S,8R,9S,10S,13S,14S,17S)-3-hydroxy-3,10,13-trimethyl-1,2,4,5,6,7,8,9,11,12,14,15,16,17-tetradecahydrocyclopenta[α]phenanthren-17-yl]ethanone), alphaxolone, alphadolone, hydroxydione, minaxolone, and Althesin (a mixture of alphaxolone, alphadolone, tetrahydrodeoxycorticosterone, pregnenolone, dehydroepiandrosterone (DHEA), 7-substituted benz[e]indene-3-carbonitriles (see, e.g., Hu, et al., J Med. Chem. (1993) 36(24):3956-67); 7-(2-hydroxyethyl)benz[e]indene analogues (see, e.g., Han, et al., J Med. Chem. (1995) 38(22):4548-56); 3 alpha-hydroxy-5 alpha-pregnan-20-one and 3 alpha-hydroxy-5 beta-pregnan-20-one analogues (see, e.g., Han, et al., J Med. Chem. (1996) 39(21):4218-32); enantiomers of dehydroepiandrosterone sulfate, pregnenolone sulfate, and (3alpha,5beta)-3-hydroxypregnan-20-one sulfate (see, e.g., Nilsson, et al., J Med. Chem. (1998) 41(14):2604-13); 13,24-cyclo-18,21-dinorcholane analogues (see, e.g., Jiang, et al., J Med. Chem. (2003) 46(25):5334-48); N-acylated 17α-aza-D-homosteroid analogues (see, e.g., Covey, et al., J Med. Chem. (2000) 43(17):3201-4); 5 beta-methyl-3-ketosteroid analogues (see, e.g., Zeng, et al., J Org. Chem. (2000) 65(7):2264-6); 18-norandrostan-17-one analogues (see, e.g., Jiang, et al., J Org. Chem. (2000) 65(11):3555-7); (3alpha,5alpha)- and (3alpha,5beta)-3-hydroxypregnan-20-one analogs (see, e.g., Zeng, et al., J Med. Chem. (2005) 48(8):3051-9); benz[f]indenes (see, e.g., Scaglione, et al., J Med. Chem. (2006) 49(15):4595-605); enantiomers of androgens (see, e.g., Katona, et al., Eur J Med. Chem. (2008) 43(1):107-13); cyclopenta[b]phenanthrenes and cyclopenta[b]anthracenes (see, e.g., Scaglione, et al., J Med. Chem. (2008) 51(5):1309-18); 2beta-hydroxygonane derivatives (see, e.g., Wang, et al., Tetrahedron (2007) 63(33):7977-7984); Δ16-alphaxalone and corresponding 17-carbonitrile analogues (see, e.g., Bandyopadhyaya, et al., Bioorg Med Chem. Lett. (2010) 20(22):6680-4); Δ(16) and Δ(17(20)) analogues of Δ(16)-alphaxalone (see, e.g., Stastna, et al., J Med. Chem. (2011) 54(11):3926-34); neurosteroid analogs developed by CoCensys (now Purdue Neuroscience) (e.g., CCD-3693, Co2-6749 (a.k.a., GMA-839 and WAY-141839); neurosteroid analogs described in U.S. Pat. No. 7,781,421 and in PCT Patent Publications WO 2008/157460; WO 1993/003732; WO 1993/018053; WO 1994/027608; WO 1995/021617; WO 1996/016076; WO 1996/040043, as well as salts, hemisuccinates, nitrosylated, sulfates and derivatives thereof.

Illustrative benzodiazepines that find use include without limitation bretazenil, clonazepam, cloxazolam, clorazepate, diazepam, fludiazepam, flutoprazepam, lorazepam, midazolam, nimetazepam, nitrazepam, phenazepam, temazepam and clobazam. In some embodiments, the methods involve intrapulmonary administration of midazolam.

5. Formulation and Administration

The benzodiazepines, including midazolam, for use in the present methods are formulated for intrapulmonary administration. In various embodiments, the benzodiazepines are formulated for delivery via an inhaler.

In various embodiments, the benzodiazepines are nebulized. Methods and systems for intrapulmonary delivery of benzodiazepines are known in the art and find use. Illustrative systems for aerosol delivery of benzodiazepines by inhalation are described, e.g., in U.S. Pat. Nos. 5,497,763; 5,660,166; 7,060,255; and 7,540,286; and U.S. Patent Publication Nos. 2003/0032638; and 2006/0052428, each of which are hereby incorporated herein by reference in their entirety for all purposes. Preferably, the benzodiazepines are nebulized without the input of heat.

For administration of the nebulized and/or aerosolized benzodiazepine (e.g., midazolam), the size of the aerosol particulates can be within a range appropriate for intrapulmonary delivery, particularly delivery to the distal alveoli. In various embodiments, the aerosol particulates have a mass median aerodynamic diameter (“MMAD”) of less than about 5 μm, 4 μm, 3 μm, for example, ranging from about 1 μm to about 3 μm, e.g., from about 2 μm to about 3 μm, e.g., ranging from about 0.01 μm to about 0.10 μm. Aerosols characterized by a MMAD ranging from about 1 μm to about 3 μm can deposit on alveoli walls through gravitational settling and can be absorbed into the systemic circulation, while aerosols characterized by a MMAD ranging from about 0.01 μm to 0.10 μm can also be deposited on the alveoli walls through diffusion. Aerosols characterized by a MMAD ranging from about 0.15 μm to about 1 μm are generally exhaled. Thus, in various embodiments, aerosol particulates can have a MMAD ranging from 0.01 μm to about 5 μm, for example, ranging from about 0.05 μm to about 3 μm, for example, ranging from about 1 μm to about 3 μm, for example, ranging from about 0.01 μm to about 0.1 μm. The nebulized and/or aerosolized benzodiazepines, including midazolam, can be delivered to the distal alveoli, allowing for rapid absorption and efficacy.

In various embodiments, the benzodiazepine, including midazolam, is formulated in a solution comprising excipients suitable for aerosolized intrapulmonary delivery. The solution can comprise one or more pharmaceutically acceptable carriers and/or excipients. Pharmaceutically acceptable refers to approved or approvable by a regulatory agency of the Federal or a state government or listed in the U.S Pharmacopoeia or other generally recognized pharmacopoeia for use in animals, and more particularly in humans. Preferably, the solution is buffered such that the solution is in a relatively neutral pH range, for example, a pH in the range of about 4 to 8, for example, a pH in the range of about 5-7. In some embodiments, the benzodiazepine is formulated in a buffered solution, for example, phosphate-buffered saline.

In various embodiments, the benzodiazepine, including midazolam, is prepared as a concentrated aqueous solution. Ordinary metered dose liquid inhalers have poor efficiency for the delivery to the deep lung because the particle size is not sufficiently small (Kim et al., 1985 Am Rev Resp Dis 132:137-142; and Farr et al., 1995 Thorax 50:639-644). These systems are therefore used mostly for local delivery of drugs to the pulmonary airways. In addition, metered doses inhalers may not be able to deliver sufficient volumes of even a concentrated midazolam solution to produce the desired rapid antiseizure effect. Accordingly, in various embodiments, a metered doses inhaler is not used for delivery of the benzodiazepine, e.g., midazolam. In one embodiment a nebulization system with the capability of delivering <5 μm particles (e.g., the PARI LC Star, which has a high efficiency, 78% respirable fraction 0.1-5 μm. see, e.g., pari.com) is used for intrapulmonary administration. Electronic nebulizers which employ a vibrating mesh or aperture plate to generate an aerosol with the required particle size can deliver sufficient quantities rapidly and find use (See, e.g., Knoch and Keller, 2005 Expert Opin Drug Deliv 2: 377-390). Also, custom-designed hand-held, electronic nebulizers can be made and find use.

Aerosolized delivery of benzodiazepines allows for reduced dosing to achieve desired efficacy, e.g., in comparison to intravenous or intranasal delivery. Appropriate dosing will depend on the size and health of the patient and can be readily determined by a trained clinician. Initial doses are low and then can be incrementally increased until the desired therapeutic effect is achieved with little or no adverse side effects. In various embodiments, the benzodiazepines are administered via the intrapulmonary route at a dose that is about 10%, 15%, 25%, 50% or 75% of established doses for their administration via other routes (e.g., via oral, intravenous or intranasal administration). In some embodiments, the benzodiazepines, including midazolam, are administered via the intrapulmonary route at a dose in the range of about 0.05 mg/kg to about 1.0 mg/kg, for example, about 0.2 mg/kg to about 0.8 mg/kg, for example, about 0.05 mg/kg, 0.08 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1.0 mg/kg. In some embodiments, the benzodiazepines, including midazolam, are administered via the intrapulmonary route at a dose in the range of about 10 μg/kg to about 80 μg/kg, for example, about 20 μg/kg to about 60 μg/kg, for example, about 25 μg/kg to about 50 μg/kg, for example, about 10 μg/kg, 15 μg/kg, 20 μg/kg, 25 μg/kg, 30 μg/kg, 35 μg/kg, 40 μg/kg, 45 μg/kg, 50 μg/kg, 60 μg/kg, 70 μg/kg, or 80 μg/kg. In some embodiments, the benzodiazepines, including midazolam, are administered via the intrapulmonary route at a dose in the range of about 0.3 μg/kg to about 3.0 μg/kg.

6. Monitoring Efficacy

Intrapulmonary delivery of a benzodiazepine to a subject results in the prevention of the occurrence of an impending seizure and/or the rapid termination or abortion of a seizure in progress.

In various embodiments, efficacy can be monitored by the subject. For example, in a subject experiencing aura or receiving a warning from a seizure prediction device, the subject can self-administer via the intrapulmonary route a dose of the benzodiazepine. If the benzodiazepine is administered in an efficacious amount, the sensation of aura should subside and/or the seizure prediction device should no longer predict the imminent occurrence of an impending seizure. If the sensation of aura does not subside and/or the seizure prediction device continues to predict an impending seizure, a second dose of benzodiazepine can be administered.

In other embodiments, the efficacy is monitored by a caregiver. For example, in a subject experiencing the onset of a seizure or in situations where a seizure has commenced, the subject may require intrapulmonary administration of the benzodiazepine by a caregiver. If the benzodiazepine is administered in an efficacious amount, the seizure, along with the subject's symptoms of the seizure, should rapidly terminate or abort. If the seizure does not terminate, a second dose of the benzodiazepine can be administered.

### EXAMPLES

The following examples are offered to illustrate, but not to limit the claimed invention.

### Example 1

The following example demonstrates intratracheal administration of midazolam in diverse animal models of seizure in mice. The pentylenetetrazol, i.p. and i.v. seizure test, picrotoxin-induced seizures, and kainic acid induced seizure models in mice were used.

### Materials and Methods

Animals:

Male NIH Swiss mice (22-30 g) were kept in a vivarium under controlled environmental conditions (temperature, 22-26° C.; humidity, 40-50%) with an artificial 12-h light/dark cycle. Wood chips were used in all cages. Experiments were performed during the light phase of the light/dark cycle after a minimum 30-min period of acclimation to the experimental room. The animal facilities were fully accredited by the Association for Assessment and Accreditation of Laboratory Animal Care. All studies were performed under protocols approved by the Animal Care and Use Committee of the University of California, Davis in strict compliance with the Guide for the Care and Use of Laboratory Animals of the National Research Council (National Academy Press, Washington, D.C.).

Intratracheal Drug Delivery.

Intratracheal administration of midazolam was carried out as described by Oka et al. (2006). In brief, mice were briefly anesthetized using isoflurane anesthesia (4% isoflurane). The animals were immediately placed on a surgical board held at a 60° angle. The animal's mouth was kept open by hanging the upper incisors on a hook to facilitate detection of the epiglottis. An operating light was used to illuminate the view of the pharynx after displacement of the tongue with a spatula. A syringe fitted with a blunted 24-gauge/25-mm was pushed against the soft palate to enter the trachea past the vocal cords. When the tracheal cartilage ring is felt, the needle is considered properly placed within the tracheal lumen. The needle was inserted almost to the bottom of the trachea. When the animals began to show signs that the anesthesia was waning, the liquid sample was gently injected at a dose volume of 0.25 ml/kg. After the injection of solution, air (1 ml syringe) was blown inside the lungs to uniformly distribute the drug in the lungs. Following the injection, the needle was gently removed and animal was held vertically for 2 min to facilitate the downward movement of liquid in the lungs. The animal was allowed 10 min to recover fully from the anesthesia as confirmed by normal spontaneous exploratory behavior. Seizure testing then commenced. For each of the seizure tests, we verified that there was no residual effect of the anesthetic on responsiveness.

PTZ-Induced Convulsions in Mice.

PTZ was administered intraperitoneally at a dose of 80 mg/kg, which causes clonic convulsions in >97% of mice (Dhir et al., 2006). Animals were observed for a period of 30 min following injection. The time of onset of myoclonic jerks, clonus and tonic extension; and the incidence of lethality was recorded.

PTZ and Picrotoxin Seizure Threshold Tests in Mice.

The thresholds for various behavioral seizure stages induced by the GABA receptor antagonists PTZ and picrotoxin were determined by infusing the convulsant drugs via a 27 gauge-¾ inch “butterfly” needle inserted into the lateral tail vein. The needle was secured to the tail vein by a narrow piece of adhesive tape and the animal was permitted to move freely inside an inverted 2 litre glass beaker with free aeration from the top. PTZ (10 mg/ml) (Akula et al., 2008), and picrotoxin (1 mg/ml) (Chan et al., 2006) was infused at a constant rate of 0.5 ml/min using a Beckton Dickinson (1 ml) syringe mounted on an infusion pump (Model ‘11’ plus syringe pump; Harvard Apparatus, Holliston, Mass., USA). The syringe was connected to the needle by polyethylene tubing. The infusion was stopped at 3 min or at the onset of tonic extension, whichever occurred first. The thresholds to the following endpoints were determined: (i) the first myoclonic jerk; (ii) the onset of generalized clonus with loss of righting reflexes; and (iii) the onset of tonic extension. Latencies were measured from the start of convulsant infusion to the onset of all these three events. The threshold value (mg/kg) for each endpoint was determined according to the following formula: (infusion duration [sec]×infusion rate [ml/min]×convulsant drug concentration [mg/ml]×1000)/(60 [sec]×weight of mouse [g]).

Kainic Acid Seizure Threshold Test in Mice.

Threshold determinations for the excitatory amino acid agonists kainic acid (7.5 mg/ml; infusion rate 0.15 ml/min) (Kaminski et al., 2005) were obtained according to the same protocol as described for the GABA receptor antagonists.

Data Analysis.

Results are expressed as mean±S.E.M.; the significance of the difference in the responses of treatment groups with respect to control is based on one-way analysis of variance (ANOVA) followed by specific post-hoc comparisons using Tukey's test. Differences were considered statistically significant when the probability of error was less than 0.05 (P<0.05). Control values in the threshold tests were the mean threshold values for vehicle-treated groups.

### Results

Comparison of Intraperitoneal and Intratracheal Midazolam on PTZ-Induced Seizures in Mice.

Intraperitoneal PTZ (80 mg/kg) induced clonic seizures, tonic seizures and mortality in all vehicle pretreated animals (Table 1). When administered 10 min prior to PTZ, intraperitoneal midazolam (750-1000 μg/kg) provided marked seizure protection against clonic seizures and completely prevented tonic seizures and the mortality that invariably accompanies tonic seizures. Midazolam at 500 μg/kg intraperitoneal injection prevented the mice against tonic extensor but not clonic phases of PTZ seizures. Midazolam at 100 μg/kg intraperitoneal injection demonstrated slight but significant protection against tonic extensor phase of PTZ seizures. At lower doses, intratracheal midazolam administered 10 and 7 min before PTZ provided significant seizure protection. The effect of intratracheal midazolam was dose-dependent: the 12.5 μg/kg dose failed to provide complete protection against tonic seizures and mortality whereas the 100 μg/kg dose protected all animals in both the 10 and 7 min pretreatment time experiments.

TABLE 1
Comparison of intraperitoneal and intratracheal midazolam
on PTZ-induced convulsions in mice

| | | | | | | |
|---|---|---|---|---|---|---|
| | | | Percent | | | |
| | | Pre- | showing | Percent | Percent | |
| | | treatment | myoclonic | showing | showing | |
| S. No. | Treatment - Dose | time (min) | jerks | clonus | extensor | Mortality |

Intraperitoneal

| | | | | | | |
|---|---|---|---|---|---|---|
| 1 | Vehicle (10 ml/kg) | 10 | 100 | 100 | 100 | 100 |
| 2 | Midazolam (100 μg/kg) | 10 | 100 | 100 | 66.67* | 66.67* |
| 3 | Midazolam (500 μg/kg) | 10 | 100 | 83.33 | 0* | 0* |
| 4 | Midazolam (750 μg/kg) | 10 | 83.33 | 33.33* | 0* | 0* |
| 5 | Midazolam (1000 μg/kg) | 10 | 50* | 0* | 0* | 0* |

Intratracheal

| | | | | | | |
|---|---|---|---|---|---|---|
| 6 | Vehicle (0.25 ml/kg) | 10 | 100 | 100 | 100 | 100 |
| 7 | Midazolam (12.5 μg/kg) | 10 | 100 | 100 | 83.33 | 83.33 |
| 8 | Midazolam (25 μg/kg) | 10 | 100 | 100 | 50* | 50* |
| 9 | Midazolam (50 μg/kg) | 10 | 83.33 | 66.66* | 16.66* | 16.66* |
| 10 | Midazolam (100 μg/kg) | 10 | 50* | 50* | 0* | 0* |
| 11 | Midazolam (200 μg/kg) | 10 | 0* | 0* | 0* | 0* |

Intratracheal

| | | | | | | |
|---|---|---|---|---|---|---|
| 12 | Vehicle (0.25 ml/kg) | 7 | 100 | 100 | 100 | 100 |
| 13 | Midazolam (25 μg/kg) | 7 | 100 | 83.33 | 0* | 0* |
| 14 | Midazolam (50 μg/kg) | 7 | 66.66* | 50* | 0* | 0* |
| 15 | Midazolam (100 μg/kg) | 7 | 0* | 0* | 0* | 0* |

PTZ was administered at a dose of 80 mg/kg, i.p.
The second column indicates the pretreatment interval between midazolam administration and the subsequent PTZ injection.
The solution volume for intraperitoneal midazolam was 10 ml/kg and for intratracheal midazolam was 0.25 ml/kg.
Values indicate percent of animals exhibiting indicated seizure sign or mortality.
*P < 0.05 as compared to vehicle treated control group (ANOVA followed by Tukey's test).

Comparison of Intraperitoneal and Intratracheal Midazolam in the PTZ Threshold Test in Mice.

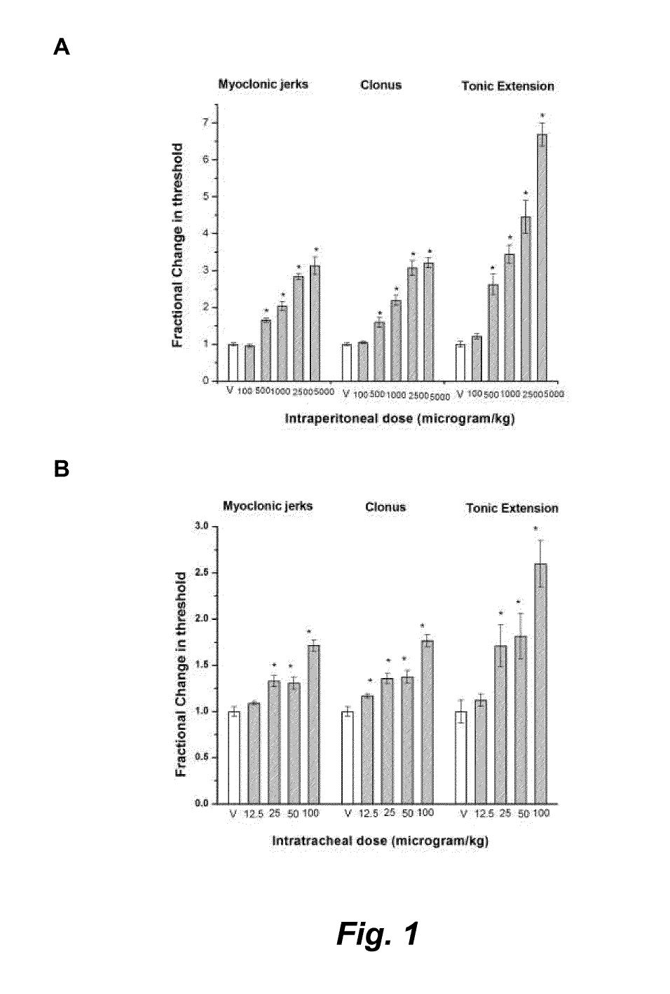

Intravenous infusion of PTZ (10 mg/ml) produced a sequence of myoclonic jerks, clonus and tonic extension followed by death. Pretreatment (15 min) with midazolam by the intraperitoneal route at doses of 500, 1000, 2500 and 5000 μg/kg but not 100 μg/kg caused a significant elevation in the thresholds for all seizure signs with respect to the values in vehicle-treated animals (FIG. 1A). In contrast, pretreatment (10 min) with midazolam by the intratracheal route was effective at lower doses of 25, 50 and 100 μg/kg (FIG. 1B). However, intratracheal administration of midazolam at 12.5 μg/kg enhanced the seizure threshold for clonic but ineffective against tonic extensor phases of convulsion.

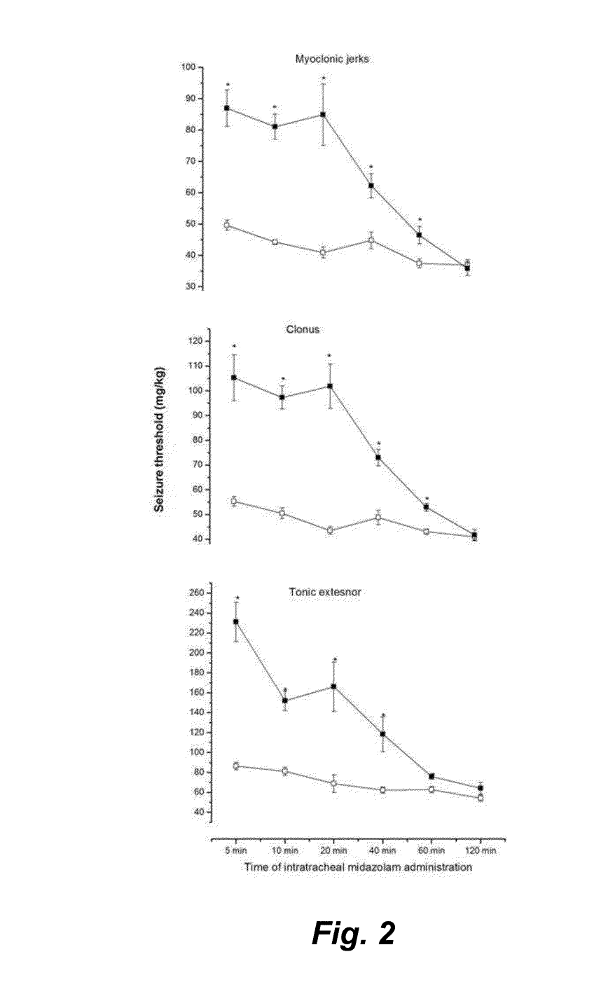

Having established that a 100 μg/kg intratracheal dose of midazolam was effective with a 10 min pretreatment time, we next sought to assess the time course of action of midazolam administered by the intratracheal route. As shown in FIG. 2, an elevation in threshold with respect to the corresponding threshold values in vehicle pretreated animals was obtained with 100 μg/kg intratracheal midazolam at 5, 10, 20, 40 and 60 min but not 120 min. The maximal effect of intratracheal midazolam occurs in between 5-15 min. It was not practical to assess shorter pretreatment times due to the confounding effect of the isoflurane anesthesia that was required for intratracheal cannulation. Indeed, the small but non-significant increase in mean threshold values obtained at the 5 min time point in the vehicle pretreatment group is likely due to residual anesthesia effects.

Motor Toxicity Testing.

The horizontal screen test was used to assess the acute motor toxicity of intratracheal midazolam (100 μg/kg) in the time course experiment. No motor toxicity was observed with midazolam at any time point.

Comparison of Intraperitoneal and Intratracheal Midazolam in the Picrotoxin Seizure Threshold Test in Mice.

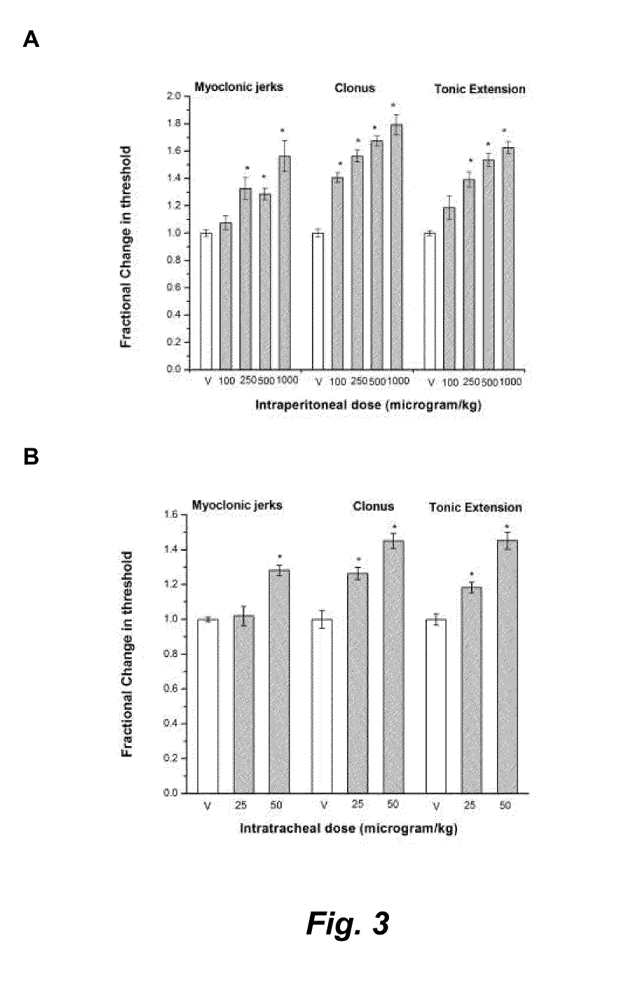

Intravenous infusion of picrotoxin (1 mg/ml) also produced the same sequence of seizure signs. Pretreatment (10 min) with midazolam by the intraperitoneal route at dose of 250, 500 and 1000 μg/kg, but not 100 μg/kg, caused a significant elevation in the thresholds for all seizure signs (FIG. 3A). A dose of 100 μg/kg administered via intraperitoneal route was effective in enhancing the seizure threshold for only clonic phase and ineffective against tonic extension of picrotoxin-induced seizure. In contrast, pretreatment (10 min) with midazolam by the intratracheal route was effective at lower doses of 25 and 50 μg/kg (FIG. 3B).

Effects of Intratracheal Administration of Midazolam in the Kainic Acid Seizure Threshold Test in Mice.

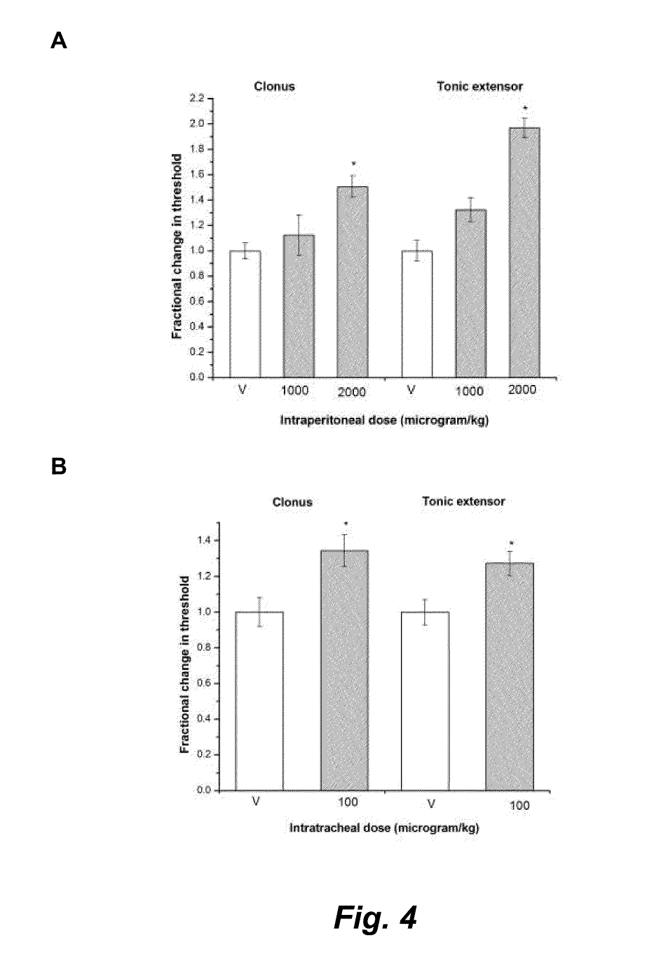

Intravenous infusion of the excitatory amino acid agonists kainic acid (7.5 mg/ml) induced a similar sequence of seizure signs as did the GABA_{A} receptor antagonists PTZ and picrotoxin. Pretreatment (10 min) with midazolam by the intraperitoneal route at dose of 2000 μg/kg but not 1000 μg/kg caused a significant elevation in the thresholds for clonus and tonic extensor phases induced due to kainic acid (FIG. 4A). In contrast, pretreatment (10 min) with midazolam by the intratracheal route was effective at lower doses of 100 μg/kg (FIG. 4B).

Intrapulmonary midazolam provides potent and rapid seizure protection indicating that intrapulmonary midazolam enters the alveoli and is rapidly absorbed into the blood stream and delivered to the brain. Administration of midazolam, or another anticonvulsant benzodiazepine, by inhalation finds use for the rapid amelioriation, termination or abortion of seizures. The pulmonary route of administration offers advantages of accelerated delivery and efficacy over the intranasal delivery.

### Example 2

### Role of Neurosteroids in the Anticonvulsant Activity of Midazolam

### Experimental Procedures

Animals.

NIH Swiss mice (22-30 g) were kept in a vivarium under controlled environmental conditions (temperature, 22-26° C.; humidity, 40-50%) with an artificial 12-h light/dark cycle. Wood chips were used in all cages. Experiments were performed during the light phase of the light/dark cycle after a minimum 30-min period of acclimation to the experimental room. The animal facilities were fully accredited by the Association for Assessment and Accreditation of Laboratory Animal Care. All studies were performed under protocols approved by the Animal Care and Use Committee of the University of California, Davis in strict compliance with the Guide for the Care and Use of Laboratory Animals of the National Research Council (National Academy Press, Washington, D.C.; on the internet at nap.edu/readingroom/books/labrats/).

Test Substances.

Midazolam was administered as a commercially available injectable solution (midazolam hydrochloride, 10 mg/10 ml; APP Pharmaceuticals, Schaumburg, Ill., USA) containing sodium chloride (0.8% w/v), disodium edetate (0.01% w/v), benzyl alcohol as preservative (1% v/v) with HCl to adjust the pH 3-3.6. Pentylenetetrazol (PTZ) (Sigma-Aldrich, St. Louis, Mo., USA) was dissolved in 0.9% w/v saline. The remaining test substances were all obtained from Tocris Bioscience (Ellisville, Mo., USA) except for clonazepam, which was from Sigma-Aldrich. Finasteride was dissolved in 25% hydroxypropyl-β-cyclodextrin (Trappsol; Cyclodextrin Technologies Development, High Springs, Fla., U.S.A.). Metyrapone was dissolved in double distilled water and PK 11195 and clonazepam were suspended in 1% Tween 80 and adjusted to volume with saline.

PTZ Seizure Threshold Test.

PTZ seizure threshold was determined according to a protocol used previously in our laboratory (Dhir et al., 2011). Intravenous PTZ elicits a sequence of seizure signs beginning with twitch and progressing to clonus and tonic limb extension. In the present study, tonic extension was used as the endpoint. In preliminary experiments, tonic extension was found to be more sensitive to midazolam than the twitch or clonus phases. It is noteworthy that this is the endpoint originally used in studies characterizing the actions of midazolam against PTZ seizures in mice (Pieri, 1983 Br J Clin Pharmacol 16 Suppl 1: 17S-27S). A 27 gauge-¾ inch “butterfly” needle was inserted into the lateral tail vein and the needle was secured with a narrow piece of adhesive tape. The animal was placed inside a 2 L glass beaker with free aeration from the top and allowed to move freely. The needle was connected by polyethylene tubing to a Beckton, Dickinson 1 ml syringe mounted on an infusion pump (Model ‘11’ plus syringe pump; Harvard Apparatus, Holliston, Mass.). PTZ solution (10 mg/ml) was infused at a constant rate of 0.5 ml/min. The infusion was stopped at 3 min or at the onset of tonic extension, whichever occurred first. The threshold value (mg/kg) for tonic extension was determined according to the formula: (infusion duration [sec]×infusion rate [ml/min]×PTZ concentration [mg/ml]×1000)/(60 [sec]×weight of mouse [g]).

Treatment Schedules.

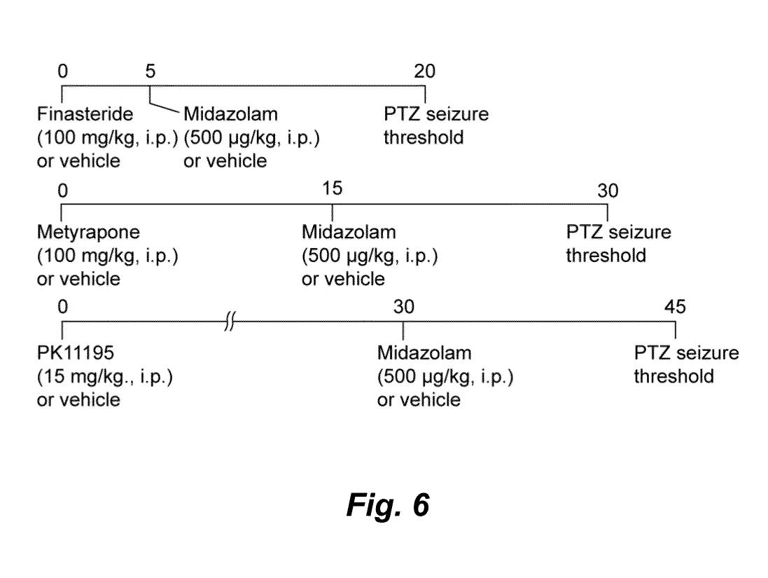

In an initial experiment, the dose-response relationship for midazolam elevation of seizure threshold was assessed at doses of 100 μg/kg to 5 mg/kg (15 min pretreatment time). The doses and time of administration of test substances in the remaining experiments with midazolam are shown in FIG. 6. In the first protocol, the 5α-reductase inhibitor finasteride was used at a dose (100 mg/kg) that has previously been shown to partially inhibit neurosteroid synthesis (Kokate et al., 1999 J Pharmacol Exp Ther 288: 679-684). Metyrapone was used at a dose (100 mg/kg) that has previously been shown to elevate seizure threshold through enhanced endogenous neurosteroid synthesis (Kaminski and Rogawski, 2011 Neuropharmacology 2011: 133-137). PK 11195 was used at a dose (15 mg/kg) as in the study of Ugale et al. (2004 Brain Res. 2004 Oct. 8; 1023(1):102-11). Clonazepam was administered at a dose of 100 μg/kg (Akula et al., 2009). The volume of all intraperitoneal injections was 10 ml/kg.

Data Analysis.

Results are expressed as mean±S.E.M.; the significance of the difference in the responses of treatment groups with respect to control is based on one-way analysis of variance (ANOVA) followed by specific post-hoc comparisons using Tukey's test. Differences are considered statistically significant when the probability of type I error was less than 0.05.

### Results

Midazolam Causes a Dose-Dependent Elevation in PTZ Seizure Threshold.

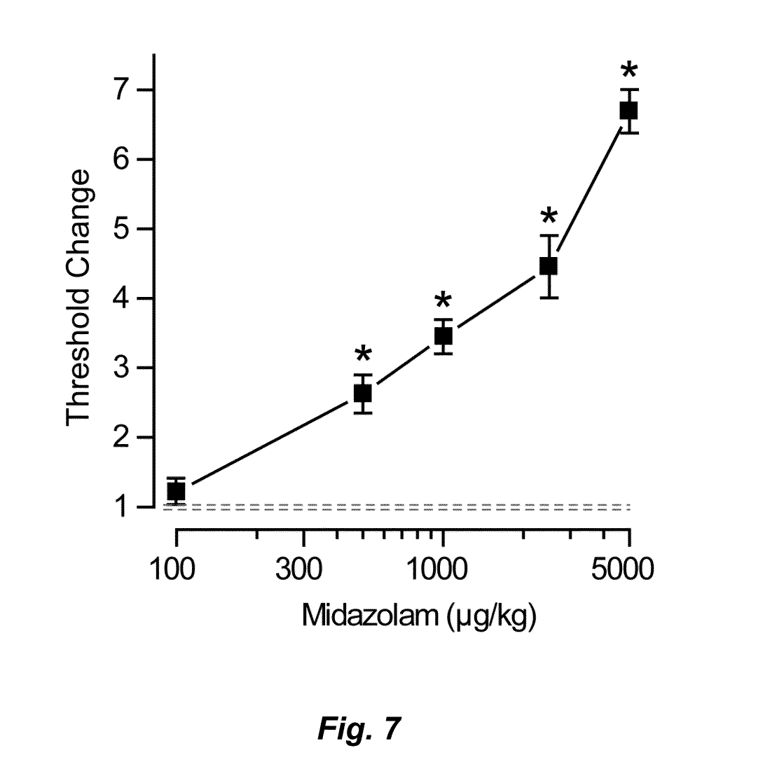

Intravenous infusion of PTZ (10 mg/ml) led to a sequence of seizure signs consisting of myoclonic jerk, clonus and tonic extension, followed by death. In the present study, cumulative dose to the onset of tonic extension was the measure of seizure threshold. Animals protected from tonic extension invariably survived whereas those that experienced tonic extension expired immediately after the seizure. FIG. 7 plots the fractional change in mean threshold for groups of animals that had been treated 15 min before the onset of the PTZ infusion with various doses of midazolam. There was a dose-dependent elevation in threshold with increasing midazolam dose that was significant for midazolam doses of 500 to 5000 μg/kg but not 100 μg/kg.

Finasteride Pretreatment Reduces the Seizure Threshold Elevation Induced by Midazolam.

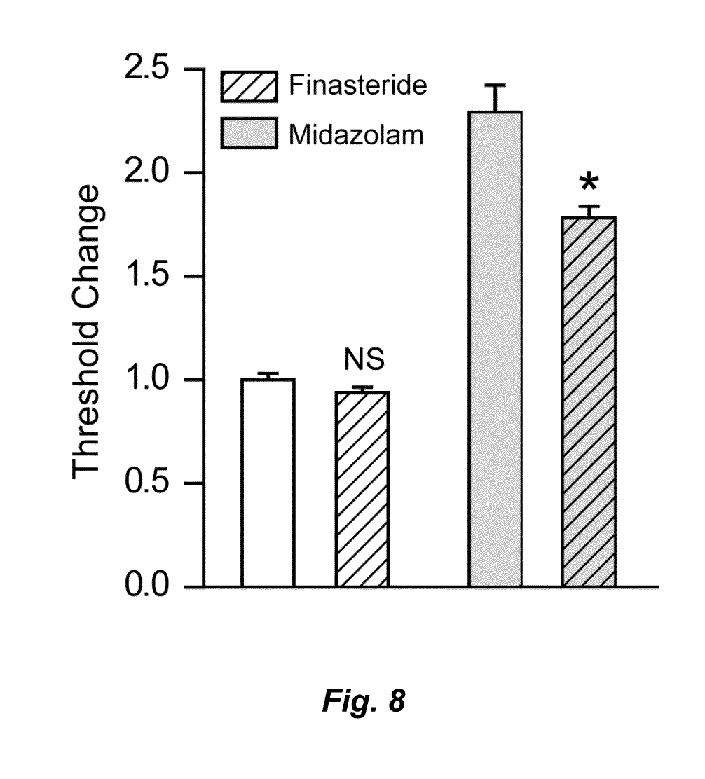

To assess whether endogenous neurosteroid production contributes to the seizure threshold elevation induced by midazolam, mice were pretreated with the neurosteroid synthesis inhibitor finasteride (100 mg/kg., i.p.) prior to administration of a dose of midazolam (500 μg/kg., i.p.) that in the experiment of FIG. 7 caused a significant increase in threshold. As shown in FIG. 8, finasteride pretreatment by itself did not alter the PTZ seizure threshold. However, finasteride did partially reduce the threshold elevation caused by midazolam.

Metyrapone Enhances the Seizure Threshold Elevation Induced by Midazolam.

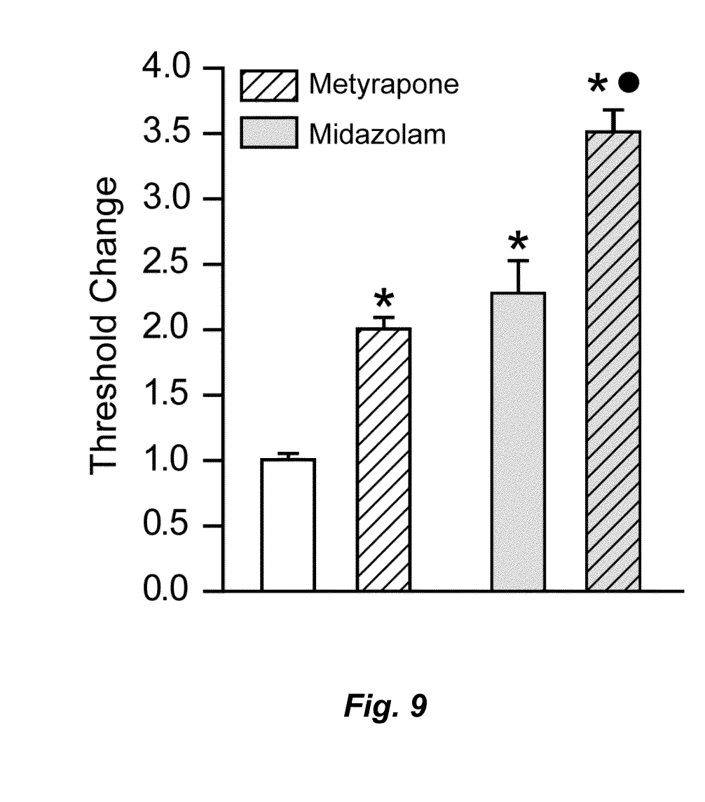

To provide further support for the involvement of neurosteroids in the anticonvulsant action of midazolam, the 11β-hydroxylase inhibitor metyrapone, which enhances endogenous neurosteroid synthesis (Rupprecht et al., 1998 Biol Psychiatry 44: 912-914; Kaminski and Rogawski, 2011 Neuropharmacology 2011: 133-137), was administered prior to midazolam. By itself, metyrapone (100 mg/kg, i.p.) caused a significant elevation in the threshold (FIG. 9), confirming our previous report (Kaminski and Rogawski, 2011 Neuropharmacology 2011: 133-137). When metyrapone was administered prior to midazolam (500 μg/kg, i.p.), there was a further increment in threshold.

PK 11195 Inhibits the Seizure Threshold Elevation Induced by Midazolam.

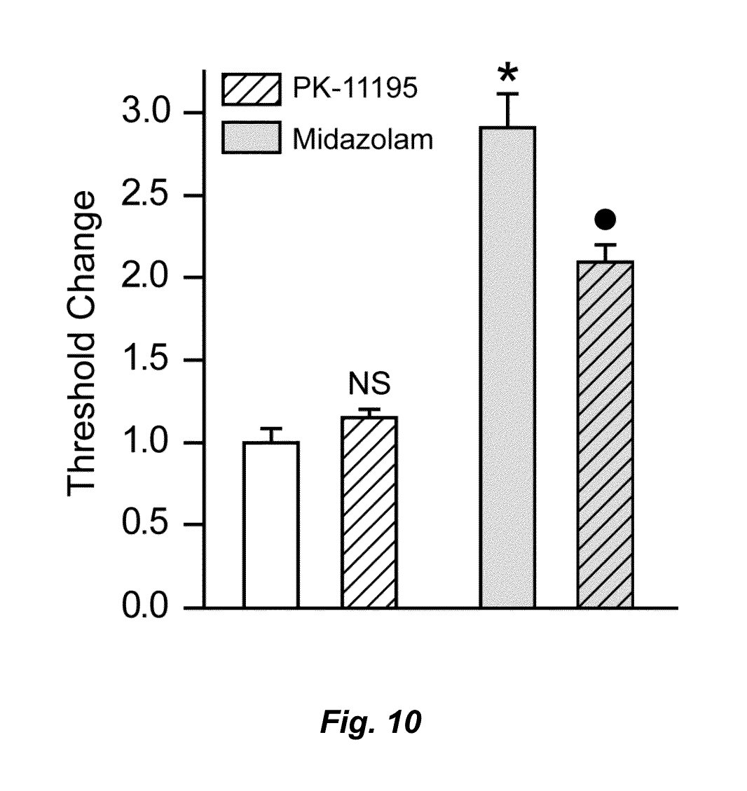

As an additional approach to assessing the role of neurosteroidogenesis in the action of midazolam we used PK 11195, a high affinity ligand of TSPO that acts as an antagonist in some situations (Le Fur et al., 1983 Life Sci 33: 449-457) and inhibits the behavioral effects of TSPO ligands that stimulate neurosteroidogenesis (Auta et al., 1993 J Pharmacol Exp Ther 265: 649-656; Romeo et al., 1993 J Pharmacol Exp Ther 267: 462-471; Frye et al., 2009 Reproduction 137: 119-128). By itself PK 11195 (15 mg/kg, i.p.) did not affect the seizure threshold. However, pretreatment with PK 11195 significantly reduced the elevation in threshold produced by midazolam (500 μg/kg, i.p.) (FIG. 10).

Effects of Finasteride and PK 11195 on the Seizure Threshold Elevation Induced by Clonazepam.

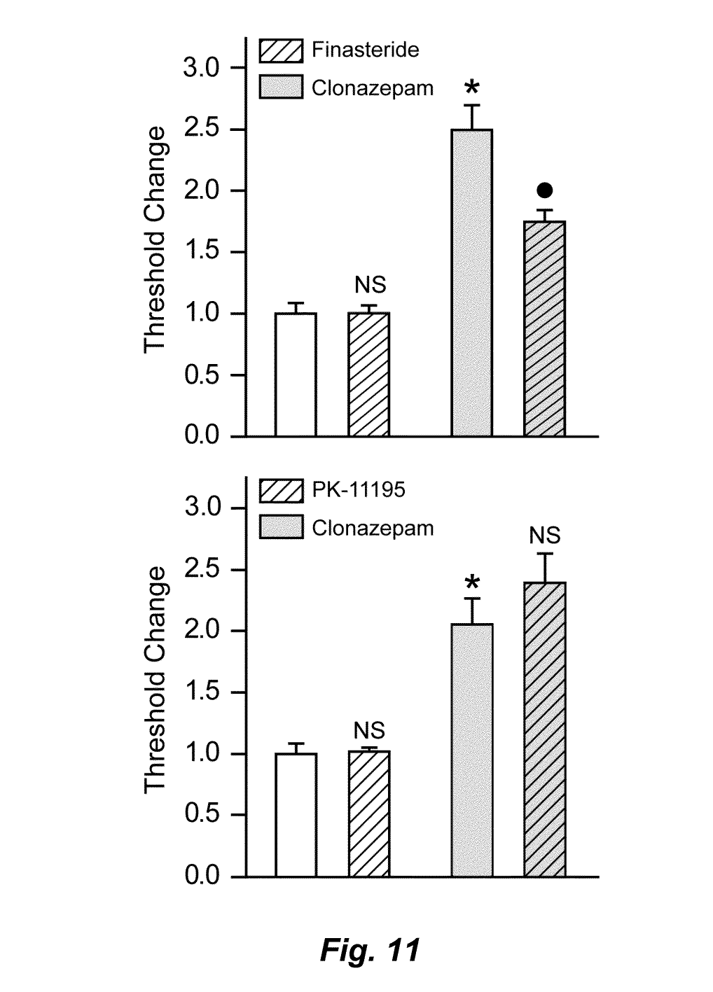

To assess the specificity of the effects of finasteride and PK 11195, a series of experiments were conducted with clonazepam, a benzodiazepine that binds only weakly to TSPO (Schoemaker et al., 1981 Eur J Pharmacol 71: 173-175; Marangos et al., 1982 Mol Pharmacol 22: 26-32; Bender and Hertz, 1987 J Neurosci Res 18: 366-372; McCauley et al., 1995 Eur J Pharmacol 276: 145-153; Guarneri et al., 1995 Brain Res 683: 65-72) and does not enhance neurosteroid synthesis in some in vitro preparations (Mukhin et al., 1989 Proc Natl Acad Sci USA 86: 9813-9816; Papadopoulos et al., 1992 Proc Natl Acad Sci USA 89: 5113-5117; Tokuda et al., 2010 J Neurosci 30: 16788-16795). As shown in FIG. 11 (upper panel), finasteride pretreatment did reduce the seizure threshold elevation produced by 100 μg/kg clonazepam. In a second experiment with a lower dose of clonazepam (25 μg/kg), there was a trend toward an effect of finasteride, although it did not reach statistical significance. However, unlike the situation with midazolam, PK 11195 did not reduce the seizure threshold elevation produced by clonazepam (FIG. 11, lower panel).

Effects of Flumazenil on the Seizure Threshold Elevation Induced by Intratracheal (i.t.) Midazolam.

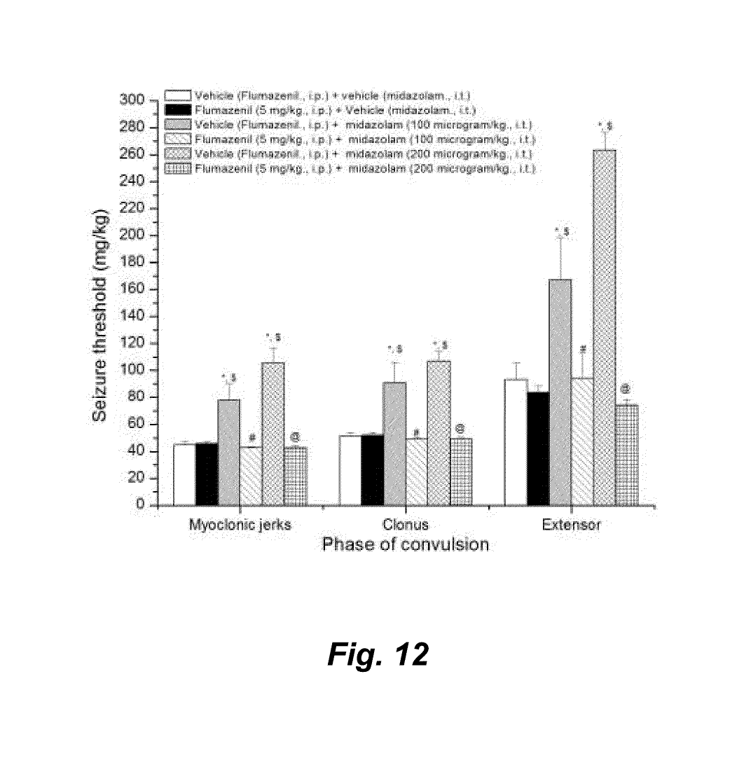

Mice were pretreated with flumazenil (5 mg/kg., i.p.), a benzodiazepine antagonist, prior to administration of midazolam (100 and 200 μg/kg., intratracheal). As shown in FIG. 12, flumazenil pretreatment by itself did not alter the PTZ seizure threshold. However, flumazenil (5 mg/kg., i.p.) did abolish the threshold elevation caused by midazolam.

Finasteride Pretreatment Reduces the Seizure Threshold Elevation Induced by Intratracheal (i.t.) Administration of Midazolam.

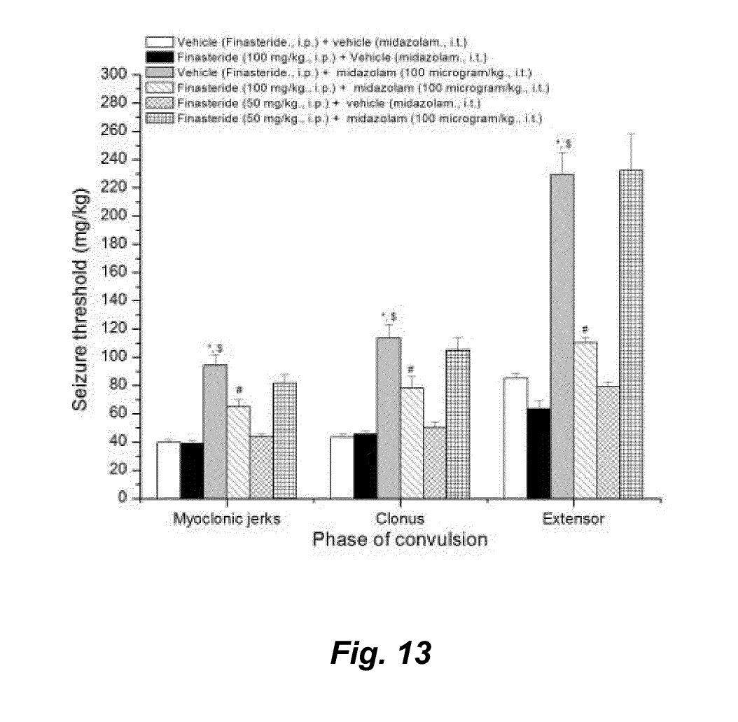

To assess whether endogenous neurosteroid production contributes to the seizure threshold elevation induced by intratracheal midazolam, mice were pretreated with the neurosteroid synthesis inhibitor finasteride (50 and 100 mg/kg., i.p.) prior to administration of a dose of midazolam (100 μg/kg., intratracheal). As shown in FIG. 13, finasteride (50 and 100 mg/kg., i.p.) pretreatment by itself did not alter the PTZ seizure threshold. However, finasteride at 100 mg/kg., i.p. did significantly reduce the threshold elevation caused by midazolam.

### DISCUSSION

This study for the first time provides evidence for the involvement of neurosteroids in the anticonvulsant activity of midazolam. As expected, midazolam caused a dose-dependent anticonvulsant action in the intravenous PTZ threshold model. The anticonvulsant activity of midazolam was significantly reduced by finasteride, a 5α-reductase inhibitor that is well recognized to suppress neurosteroidogenesis in mice (Kokate et al., 1999 J Pharmacol Exp Ther 288: 679-684; Finn et al., 2006 CNS Drug Rev 12: 53-76). For example, at the dose used in the present study, finasteride eliminates the rise in plasma allopregnanolone induced by elevation of its precursor progesterone (Reddy et al., 2001 Epilepsia 42: 328-336) and also inhibits local neurosteroid synthesis in the brain (Tokuda et al., 2010 J Neurosci 30: 16788-16795). By itself, finasteride did not affect the seizure threshold indicating that the effect on midazolam is not due to an enhancement of seizure susceptibility unrelated to the action on neurosteroidogenesis. Moreover, our result is consistent with several other studies demonstrating that finasteride does not influence basal seizure susceptibility (Reddy and Rogawski, 2002 J Neurosci 22: 3795-3805; Lawrence et al., 2010 Ann Neurol 67: 689-693), which lead to the conclusion that basal (unstimulated) neurosteroid levels do not have a tonic influence on seizure susceptibility.

In contrast to the effect of finasteride, metyrapone, an 11β-hydroxylase inhibitor, has been shown to increase neurosteroidogenesis by causing a buildup of neurosteroid precursors such as progesterone and 11-deoxycorticosterone that ordinarily flow to glucocorticoid synthesis (Kaminski et al., 2011 Neuropharmacology 2011: 133-137). In the present study, metyrapone by itself elevated the seizure threshold consistent with our previous report (Kaminski et al., 2011 Neuropharmacology 2011: 133-137). Midazolam caused a further and largely additive increment in threshold confirming that enhanced neurosteroidogenesis can augment the action of midazolam.

The TSPO ligand PK 11195 by itself did not affect PTZ seizure threshold. PK 11195 in some but not all situations acts as a TSPO antagonist (Le Fur et al., 1983 Life Sci 33: 449-457; Mizoule et al., 1985 Life Sci 36: 1059-1068; Matsumoto et al., 1994 Antimicrob Agents Chemother 38: 812-816). As such it inhibits TSPO agonist induced steroidogenesis (Cavallaro et al., 1992 Proc Natl Acad Sci USA 89: 10598-10602). PK 11195 by itself has variable effects on basal steroidogenesis. In some situations it has no effect consistent with a role as a TSPO antagonist (Cavallaro et al., 1992), whereas in other cases it may increase (Mukhin et al., 1989 Proc Natl Acad Sci USA 86: 9813-9816; McCauley et al., 1995 Eur J Pharmacol 276: 145-153) or decrease (Frye et al., 2009 Reproduction 137: 119-128) steroidogenesis. These latter actions could be due to weak intrinsic (partial agonist) activity or an influence on endogenous TSPO ligands. Even though PK 11195 may reduce endogenous neurosteroid levels in some circumstances, the lack of effect of PK 11195 on basal seizure threshold is consistent with the results of several previous studies with finasteride discussed above that have concluded that basal neurosteroid levels do not influence seizure susceptibility. Here we took advantage of the ability of PK 11195 to antagonize neurosteroidogenesis activated by TSPO ligands. We observed that PK 11195 caused a significant inhibition of the seizure threshold increase produced by midazolam. This provides evidence that the anticonvulsant action of midazolam depends in part on its ability to interact with TSPO as an agonist.

To further explore the role of TSPO, we conducted experiments with clonazepam, a benzodiazepine that is a very weak TSPO ligand. Surprisingly, we observed that the seizure threshold increase produced by clonazepam was reduced by finasteride. This occurred at a higher dose of clonazepam but not at a lower dose. Given the weak affinity of clonazepam for TSPO, the concentrations achieved in brain with either dose are unlikely to produce substantial occupancy of TSPO. Accordingly, PK 11195 failed to reduce the effect of clonazepam on seizure threshold demonstrating that its activity is not mediated by TSPO. At present, the basis through which finasteride inhibits the response to clonazepam is uncertain. There are no known pharmacokinetic interactions between finasteride and benzodiazepines, including clonazepam and midazolam. While clonazepam does not stimulate neurosteroid synthesis in mitochondria (Papadopoulos et al., 1992 Proc Natl Acad Sci USA 89: 5113-5117), isolated cell systems (Mukhin et al., 1989 Proc Natl Acad Sci USA 86: 9813-9816) or some brain regions (Tokuda et al., 2010 J Neurosci 30: 16788-16795), in at least one region of the central nervous system (retina) it can potently and rapidly (within minutes) enhance neurosteroid synthesis (Guarneri et al., 1995 Brain Res 683: 65-72). This effect of clonazepam, which occurs in a PK 11195-independent fashion, appears to be mediated by a direct interaction with GABA_{A} receptors. Whether a similar action occurs in brain regions relevant to the anticonvulsant activity of clonazepam remains to be determined.

In conclusion, our results demonstrate a role for neurosteroids in the anticonvulsant action of midazolam. We propose that in addition to directly activating GABA_{A} receptors through an agonist interaction with the intrinsic benzodiazepine recognition site, midazolam enhances neurosteroid synthesis through an agonist interaction with TSPO although we cannot exclude the possibility that this occurs in part through a direct interaction with GABA_{A} receptors as is likely the case for clonazepam. Whether the enhanced neurosteroidogenesis occurs peripherally or directly in the brain is not defined in the present study. Although there is evidence that midazolam can influence neurosteroid synthesis locally in the brain (Tokuda et al., 2010 J Neurosci 30: 16788-16795), neurosteroids synthesized peripherally can readily enter the brain to influence seizure susceptibility. Therefore, enhanced peripheral neurosteroid synthesis could contribute to the neurosteroid-related component of the effect of midazolam on seizure threshold noted in the present study. Neurosteroids are known to bind to distinct sites on GABA_{A} receptors through which they cause positive allosteric modulation of GABA responses (at low concentrations) and direct activation of the receptor (at higher concentrations) (Hosie et al., 2007 Pharmacol Ther 116: 7-19). Unlike neurosteroids, agonists that act at the benzodiazepine recognition site do not directly activate GABA_{A} receptors in the absence of GABA. Moreover, neurosteroids cause markedly greater maximal potentiation of GABA responses than do benzodiazepine recognition site agonists (Kokate et al., 1994 J Pharmacol Exp Ther. 270: 1223-1229). The capacity of neurosteroids to cause large magnitude positive modulation of GABA responses and also to directly activate GABA_{A} receptors confers neurosteroids with potent anticonvulsant properties (Reddy and Rogawski, 2004 J Neurosci 22: 3795-3805). The combination of the direct action of midazolam on synaptic GABA_{A} receptors along with the indirect actions mediated by neurosteroids could account for the particularly effective anticonvulsant action of midazolam (Raines et al., 1990 Epilepsia 31: 313-317). Benzodiazepines only act on a restricted subset of GABA_{A} receptor isoforms (Olsen and Sieghart, 2008 Pharmacol Rev 60: 243-260). Neurosteroids, in contrast, act on all GABA_{A} receptors subunit combinations and produce a particularly large augmentation in the activity of certain non-synaptic forms, such as those containing 6 subunits, that mediate tonic inhibition (Stell et al., 2003 Proc Natl Acad Sci USA 100: 14439-14444; Farrant and Nusser, 2005 Nat Rev Neurosci 6: 215-229). It is reasonable to speculate that an effect on non-synaptic, benzodiazepine-insensitive GABA_{A} receptors mediated indirectly by neurosteroids also contributes to the powerful anticonvulsant action of midazolam. Neurosteroids may also contribute to the anticonvulsant actions of other benzodiazepines with TSPO binding activity and there are benzodiazepines, most notably clonazepam, that may influence neurosteroids through mechanisms that do not involve TSPO.

### REFERENCES

Akula K K, Dhir A, Kulkarni S K (2008) Nitric oxide signaling pathway in the anti-convulsant effect of adenosine against pentylenetetrazol-induced seizure threshold in mice. Eur J Pharmacol 587:129-134.
Aurora S K, Rozen T D, Kori S H, and Shrewsbury S B (2009) A randomized, double blind, placebo-controlled study of MAP0004 in adult patients with migraine. Headache 49: 826-837.
Bhattacharyya M, Kalra V, Gulati S (2006). Intranasal midazolam vs rectal diazepam in acute childhood seizures. Pediatri Neurol 34: 355-359.
Chan M H, Lee C C, Chen H H (2006) Effects of toluene on seizures induced by convulsants acting at distinct ligand-gated ion channels. Toxicol Lett 160:179-184.
Czlonkowska A I, Krzaścik P, Sienkiewicz-Jarosz H, Siemiatkowski M, Szyndler J, Maciejak P, Bidziński A, Plaźniki A (2001). Tolerance to the anticonvulsant activity of midazolam and allopregnanolone in a model of picrotoxin seizures. Eur J Pharmacol 425: 121-127.
Dhir A, Naidu P S, Kulkarni S K (2006) Effect of cyclooxygenase inhibitors on pentylenetetrazol (PTZ)-induced convulsions: Possible mechanism of action. Prog Neuropsychopharmacol Biol Psychiatry 30:1478-1485.
Galvin G M, Jelinek G A (1987). Midazolam: An effective intravenous agent for seizure control. Archives of Emergency Medicine 4: 169-172.
Gilat E, Goldman M, Lahat E, Levy A, Rabinovitz I, Cohen G, Brandeis R, Amitai G, Alkalai D, Eshel G (2003). Nasal midazolam as a novel anticonvulsive treatment against organophosphate-induced seizure activity in the guinea pig. Archives of Toxicology 77: 167-172.
Jaimovich D G, Shabino C L, Noorani P A, Bittle B K, Osborne J S (1990). Intravenous midazolam suppression on pentylenetetrazol-induced epileptogenic activity in a porcine model. Crit. Care Med 18: 313-316.
Kaminski R M, Shippenberg T S, Witkin J M, Rocha B A (2005) Genetic deletion of the norepinephrine transporter decreases vulnerability to seizures. Neurosci Lett 382:51-55.
Liefaard L C, Gunput R A, Danhof M, Voskuyl R A (2007). Decreased Efficacy of GABA_{A}-receptor modulation by midazolam in the kainate model of temporal lobe epilepsy. Epilepsia 48:1378-1387.
Oka Y, Mitsui M, Kitahashi T, Sakamoto A, Kusuoka O, Tsunoda T, Mori T, Tsutsumi M (2006) A reliable method for intratracheal instillation of materials to the entire lung in rats. J Toxicol Pathol 19:107-109.
Orebaugh S L, Bradford S M (1994). Intravenous versus intramuscular midazolam in treatment of chemically induced generalized seizures in swine. Am J Emerg Med 12: 284-287.
Raines A, Henderson T R, Swinyard E A, Dretchen K L (1990). Comparison of midazolam and diazepam by the intramuscular route for the control of seizures in a mouse model of status epilepticus. Epilepsia 31:313-317.
Wieland H A, Lüddens H, Seeburg P H (1992). A single histidine in GABA_{A} receptors is essential for benzodiazepine agonist binding. J Biol Chem 267:1426-1429.
Akula K K, Dhir A, Kulkarni S K (2009). Effect of various antiepileptic drugs in a pentylenetetrazol induced seizure model in mice. Methods Find Exp Clin Pharmacol 31: 423-432.
Auta J, Romeo E, Kozikowski A, Ma D, Costa E, Guidotti A (1993). Participation of mitochondrial diazepam binding inhibitor receptors in the anticonflict, antineophobic and anticonvulsant action of 2-aryl-3-indoleacetamide and imidazopyridine derivatives. J Pharmacol Exp Ther 265: 649-656.
Bender A S, Hertz L (1987). Pharmacological characteristics of diazepam receptors in neurons and astrocytes in primary cultures. J Neurosci Res 18: 366-372.
Bitran D, Foley M, Audette D, Leslie N, Frye C A (2000). Activation of peripheral mitochondrial benzodiazepine receptors in the hippocampus stimulates allopregnanolone synthesis and produces anxiolytic-like effects in the rat. Psychopharmacology (Berl) 151: 64-71.
Cavallaro S, Korneyev A, Guidotti A, Costa E (1992). Diazepam-binding inhibitor (DBI)-processing products, acting at the mitochondrial DBI receptor, mediate adrenocorticotropic hormone-induced steroidogenesis in rat adrenal gland. Proc Natl Acad Sci USA 89: 10598-10602.
Dhir A, Zolkowska D, Murphy R B, Rogawski M A (2011). Seizure protection by intrapulmonary delivery of propofol hemisuccinate. J Pharmacol Exp Ther 336: 215-222.
Eom W, Lee J M, Park J, Choi K, Jung S J, Kim H S (2011). The effects of midazolam and sevoflurane on the GABA_{A} receptors with alternatively spliced variants of the γ2 subunit. Korean J Anesthesiol 60: 109-118.
Farrant M, Nusser Z (2005). Variations on an inhibitory theme: phasic and tonic activation of GABA_{A} receptors. Nat Rev Neurosci 6: 215-229.
Finn D A, Beadles-Bohling A S, Beckley E H, Ford M M, Gililland K R, Gorin-Meyer R E et al. (2006). A new look at the 5α-reductase inhibitor finasteride. CNS Drug Rev 12: 53-76.
Frye C A, Paris J J, Rhodes M E (2009). Increasing 3α,5α-THP following inhibition of neurosteroid biosynthesis in the ventral tegmental area reinstates anti-anxiety, social, and sexual behavior of naturally receptive rats. Reproduction 137: 119-128.
Galvin G M, Jelinek G A (1987). Midazolam: An effective intravenous agent for seizure control. Arch Emerg Med 4: 169-172.
Gavish M, Bachman I, Shoukrun R, Katz Y, Veenman L, Weisinger G et al. (1999). Enigma of the peripheral benzodiazepine receptor. Pharmacol Rev 51: 629-650.
Guarneri P, Guarneri R, Cascio C, Piccoli F, Papadopoulos V (1995). γ-Aminobutyric acid type A/benzodiazepine receptors regulate rat retina neurosteroidogenesis. Brain Res 683: 65-72.
Holloway C D, Kenyon C J, Dowie L J, Corrie J E, Gray C E, Fraser R (1989). Effect of the benzodiazepines diazepam, des-N-methyldiazepam and midazolam on corticosteroid biosynthesis in bovine adrenocortical cells in vitro; location of site of action. J Steroid Biochem 33: 219-225.
Hosie A M, Wilkins M E, Smart T G (2007). Neurosteroid binding sites on GABA_{A} receptors. Pharmacol Ther 116: 7-19.
Jaimovich D G, Shabino C L, Noorani P A, Bittle B K, Osborne J S (1990). Intravenous midazolam suppression on pentylenetetrazol-induced epileptogenic activity in a porcine model. Crit. Care Med 18: 313-316.
Kaminski R M, Rogawski M A (2011). 11β-Hydroxylase inhibitors protect against seizures in mice by increasing endogenous neurosteroid synthesis. Neuropharmacology 2011: 133-137.
Kita A, Furukawa K (2008). Involvement of neurosteroids in the anxiolytic-like effects of AC-5216 in mice. Pharmacol Biochem Behav 89: 171-178.
Kita A, Kohayakawa H, Kinoshita T, Ochi Y, Nakamichi K, Kurumiya S et al. (2004). Antianxiety and antidepressant-like effects of AC-5216, a novel mitochondrial benzodiazepine receptor ligand. Br J Pharmacol 142: 1059-1072.
Kokate T G, Banks M K, Magee T, Yamaguchi S, Rogawski M A (1999). Finasteride, a 5α-reductase inhibitor, blocks the anticonvulsant activity of progesterone in mice. J Pharmacol Exp Ther 288:679-684.
Kokate T G, Svensson B E, Rogawski M A (1994). Anticonvulsant activity of neurosteroids: correlation with γ-aminobutyric acid-evoked chloride current potentiation. J Pharmacol Exp Ther. 270: 1223-1229.
Kucken A M, Teissére J A, Seffinga-Clark J, Wagner D A, Czajkowski C (2003). Structural requirements for imidazobenzodiazepine binding to GABA(A) receptors. Mol Pharmacol 63: 289-296.
Lacapère J J, Papadopoulos V (2003). Peripheral-type benzodiazepine receptor: structure and function of a cholesterol-binding protein in steroid and bile acid biosynthesis. Steroids 68: 569-585.
Lawrence C, Martin B S, Sun C, Williamson J, Kapur J (2010). Endogenous neurosteroid synthesis modulates seizure frequency. Ann Neurol 67: 689-693.
Le Fur G, Vaucher N, Perrier M L, Flamier A, Benavides J, Renault C et al. (1983). Differentiation between two ligands for peripheral benzodiazepine binding sites, [³H]RO5-4864 and [³H]PK 11195, by thermodynamic studies. Life Sci 33: 449-457.
Mak J C, Barnes P J (1980). Peripheral type benzodiazepine receptors in human and guinea pig lung: characterization and autoradiographic mapping. J Pharmacol Exp Ther 252: 880-885.
Marangos P J, Patel J, Boulenger J P, Clark-Rosenberg R (1982). Characterization of peripheral-type benzodiazepine binding sites in brain using [³H]Ro 5-4864. Mol Pharmacol 22: 26-32.
Matsumoto T, Ogata M, Koga K, Shigematsu A (1994). Effect of peripheral benzodiazepine receptor ligands on lipopolysaccharide-induced tumor necrosis factor activity in thioglycolate-treated mice. Antimicrob Agents Chemother 38: 812-816.
McCauley L D, Park C H, Lan N C, Tomich J M, Shively J E, Gee K W (1995). Benzodiazepines and peptides stimulate pregnenolone synthesis in brain mitochondria. Eur J Pharmacol 276: 145-153.
Mizoule J, Gauthier A, Uzan A, Renault C, Dubroeucq M C, Guérémy C et al. (1985). Opposite effects of two ligands for peripheral type benzodiazepine binding sites, PK 11195 and RO5-4864, in a conflict situation in the rat. Life Sci 36: 1059-1068.
Mukhin A G, Papadopoulos V, Costa E, Krueger K E (1989). Mitochondrial benzodiazepine receptors regulate steroid biosynthesis. Proc Natl Acad Sci USA 86: 9813-9816.
Olsen R W, Sieghart W (2008). International Union of Pharmacology. LXX. Subtypes of γ-aminobutyric acid A receptors: classification on the basis of subunit composition, pharmacology, and function. Update. Pharmacol Rev 60: 243-260.
Orebaugh S L, Bradford S M (1994). Intravenous versus intramuscular midazolam in treatment of chemically induced generalized seizures in swine. Am J Emerg Med 12: 284-287.
Papadopoulos V, Baraldi M, Guilarte T R, Knudsen T B, Lacapère J J, Lindemann P et al. (2006). Translocator protein (18 kDa): new nomenclature for the peripheral-type benzodiazepine receptor based on its structure and molecular function. Trends Pharmacol Sci 27: 402-409.
Papadopoulos V, Guarneri P, Kreuger K E, Guidotti A, Costa E (1992). Pregnenolone biosynthesis in C6-2B glioma cell mitochondria: regulation by a mitochondrial diazepam binding inhibitor receptor. Proc Natl Acad Sci USA 89: 5113-5117.
Pieri L (1983). Preclinical pharmacology of midazolam. Br J Clin Pharmacol 16 Suppl 1: 17S-27S.
Raines A, Henderson T R, Swinyard E A, Dretchen K L (1990). Comparison of midazolam and diazepam by the intramuscular route for the control of seizures in a mouse model of status epilepticus. Epilepsia 31: 313-317.
Reddy D S, Kim H Y, Rogawski M A (2001). Neurosteroid withdrawal model of perimenstrual catamenial epilepsy. Epilepsia 42: 328-336.
Reddy D S, Rogawski M A (2002). Stress-induced deoxycorticosterone-derived neurosteroids modulate GABAA receptor function and seizure susceptibility. J Neurosci 22: 3795-3805.
Rogawski M A (2002). Principles of antiepileptic drug action. In, Levy R H, Mattson R H, Meldrum B S, Perucca E (eds). Antiepileptic Drugs, Fifth Edition, Lippincott Williams & Wilkins Philadelphia, pp 3-22.
Rogawski M A, Reddy D S (2004). Neurosteroids: Endogenous modulators of seizure susceptibility. In: Rho J M, Sankar R, Cavazos J (eds). Epilepsy: Scientific Foundations of Clinical Practice, Marcel Dekker: New York, pp 319-355.
Romeo E, Cavallaro S, Komeyev A, Kozikowski A P, Ma D, Polo A et al. (1993). Stimulation of brain steroidogenesis by 2-aryl-indole-3-acetamide derivatives acting at the mitochondrial diazepam binding inhibitor receptor complex. J Pharmacol Exp Ther 267: 462-471.
Rovira C, Ben-Ari Y (1993). Developmental study of benzodiazepine effects on monosynaptic GABA_{A}-mediated IPSPs of rat hippocampal neurons. J Neurophysiol 70: 1076-1085.
Rupprecht R, Rammes G, Eser D, Baghai T C, Schüle C, Nothdurfter C et al. (2009). Translocator protein (18 kD) as target for anxiolytics without benzodiazepine-like side effects. Science 325: 490-493.
Rupprecht R, Ströhle A, Hermann B, di Michele F, Spalletta G, Pasini A et al. (1998). Neuroactive steroid concentrations following metyrapone administration in depressed patients and healthy volunteers. Biol Psychiatry 44: 912-914.
Schoemaker H, Bliss M, Yamamura H I (1981). Specific high-affinity saturable binding of [³H] Ro5-4864 to benzodiazepine binding sites in the rat cerebral cortex. Eur J Pharmacol 71: 173-175.
Riisch D, Forman S A (2005). Classic benzodiazepines modulate the open-close equilibrium in α₁β₂γ_{2L} γ-aminobutyric acid type A receptors. Anesthesiology 102: 783-792.
Serra M, Madau P, Chessa M F, Caddeo M, Sanna E, Trapani G et al. (1999). 2-Phenyl-imidazo[1,2-a]pyridine derivatives as ligands for peripheral benzodiazepine receptors: stimulation of neurosteroid synthesis and anticonflict action in rats. Br J Pharmacol 127: 177-187.
So E C, Chang Y T, Hsing C H, Poon P W, Leu S F, Huang B M (2010). The effect of midazolam on mouse Leydig cell steroidogenesis and apoptosis. Toxicol Lett 192: 169-178.
Stell B M, Brickley S G, Tang C Y, Farrant M, Mody I (2003). Neuroactive steroids reduce neuronal excitability by selectively enhancing tonic inhibition mediated by δ subunit-containing GABA_{A} receptors. Proc Natl Acad Sci USA 100: 14439-14444.
Tokuda K, O'Dell K A, Izumi Y, Zorumski C F (2010). Midazolam inhibits hippocampal long-term potentiation and learning through dual central and peripheral benzodiazepine receptor activation and neurosteroidogenesis. J Neurosci 30: 16788-16795.
Tsuda M, Suzuki T, Misawa M (1998). Subsensitivity to mitochondrial diazepam binding inhibitor receptor agonist FGIN-1-27-induced antiseizure effect in diazepam-withdrawn mice. Life Sci. 62:PL213-217.
Verleye M, Akwa Y, Liere P, Ladurelle N, Pianos A, Eychenne B et al. (2005). The anxiolytic etifoxine activates the peripheral benzodiazepine receptor and increases the neurosteroid levels in rat brain. Pharmacol Biochem Behav 82: 712-720.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of preventing or terminating a seizure in a subject in need thereof, comprising intrapulmonary administration to the subject of an effective amount of a benzodiazepine receptor agonist.

2. The method of claim 1, wherein the benzodiazepine receptor agonist is a benzodiazepine.

3. The method of claim 1, wherein the benzodiazepine is an agonist of the benzodiazepine recognition site on GABA_{A} receptors and stimulates endogenous neurosteroid synthesis.

4. The method of claim 1, wherein the benzodiazepine is selected from the group consisting of bretazenil, clonazepam, cloxazolam, clorazepate, diazepam, fludiazepam, flutoprazepam, lorazepam, midazolam, nimetazepam, nitrazepam, phenazepam, temazepam and clobazam.

5. The method of claim 1, wherein the benzodiazepine is midazolam.

6. The method of claim 1, wherein the subject is experiencing aura.

7. The method of claim 1, wherein the subject has been warned of an impending seizure.

8. The method of claim 1, wherein the subject is experiencing a seizure.

9. The method of claim 1, wherein the subject has status epilepticus.

10. The method of claim 1, wherein the subject has myoclonic epilepsy.

11. The method of claim 1, wherein the subject suffers from seizure clusters.

12. The method of claim 1, wherein the seizure is a tonic seizure.

13. The method of claim 1, wherein the seizure is a clonic seizure.

14. The method of claim 1, wherein the benzodiazepine is not heated.

15. The method of claim 1, wherein the benzodiazepine is nebulized.

16. The method of claim 15, wherein the nebulized particles are about 3 μm or smaller.

17. The method of claim 15, wherein the nebulized particles are about 2-3 μm.

18. The method of claim 1, wherein the benzodiazepine is delivered to the distal alveoli.

19. The method of claim 1, wherein the benzodiazepine is administered at a dose in the range of 0.3 μg/kg to 3.0 μg/kg.

20. The method of claim 1, wherein the benzodiazepine is self-administered by the subject.

21. A method of accelerating the termination or abortion of an impending seizure in a subject in need thereof, comprising intrapulmonary administration to the subject of an effective amount of a benzodiazepine.

22-40. (canceled)
